SEQUENCE LISTING (22 sequences)

SEQ ID NO: 1 - DNA, 21 bp, Artificial Sequence
  Synthetic (Listeria_A4_F primer)
ccaagcagtgagtgtgagaag

SEQ ID NO: 2 - DNA, 20 bp, Artificial Sequence
  Synthetic construct (Listeria_A4_F1 primer)
ccaagcagtgagtgtgagaa

SEQ ID NO: 3 - DNA, 21 bp, Artificial Sequence
  Synthetic construct (Listeria_A4_F2 primer)
tccaagcagtgagtgtgagaa

SEQ ID NO: 4 - DNA, 20 bp, Artificial Sequence
  Synthetic construct (Listeria_A4_F3 primer)
ccaagcagtgagtgtgagaa

SEQ ID NO: 5 - DNA, 21 bp, Artificial Sequence
  Synthetic construct (Listeria_A4_R primer)
tgacagcgtgaaatcaggaac

SEQ ID NO: 6 - DNA, 19 bp, Artificial Sequence
  Synthetic construct (Listeria_A4_R1)
ttgacagcgtgaaatcagg

SEQ ID NO: 7 - DNA, 19 bp, Artificial Sequence
  Synthetic construct (Listeria_A4_R2)
tgacagcgtgaaatcagga

SEQ ID NO: 8 - DNA, 19 bp, Artificial Sequence
  Synthetic construct (Listeria_A4_R2D)
tgacagcgtgaaatcagga

SEQ ID NO: 9 - DNA, 18 bp, Artificial Sequence
  Synthetic construct (Listeria_A4_R3)
gacagcgtgaaatcagga

SEQ ID NO: 10 - DNA, 22 bp, Artificial Sequence
  misc_feature (9)..(9): ribonucleotide
tgcgaagcatgagctgtgatgg

SEQ ID NO: 11 - DNA, 22 bp, Artificial Sequence
  misc_feature (8)..(8): ribonucleotide
tgcgaagcatgagctgtgatgg

SEQ ID NO: 12 - DNA, 21 bp, Artificial Sequence
  misc_feature (14)..(14): ribonucleotide
ccatcacagctcaugcttcgc

SEQ ID NO: 13 - DNA, 21 bp, Artificial Sequence
  misc_feature (12)..(14): ribonucleotide
ccatcacagctcaugcttcgc

SEQ ID NO: 14 - DNA, 21 bp, Artificial Sequence
  misc_feature (11)..(14): ribonucleotide
ccatcacagcucaugcttcgc

SEQ ID NO: 15 - protein, 224 aa, Pyrococcus furiosus
MKIGGIDEAGRGPAIGPLVVATVVVDEKNIEKLRNIGVKDSKQLTPHERKNLFSQITSIADDYKIVIVSPEEIDNRSGTMNELEVEKFALALNSLQIKPALIYADAADVDANRFASLIERRLNYKAKIIAEHKADAKYPVVSAASILAKVVRDEEIEKLKKQYGDFGSGYPSDPKTKKWLEEYYKKHNSFPPIVRRTWETVRKIEESIKAKKSQLTLDKFFKKP

SEQ ID NO: 16 - DNA, 20 bp, Artificial
  Synthetic construct
acgagtaacgggacaaatgc

SEQ ID NO: 17 - DNA, 20 bp, Artificial
  Synthetic construct
tccctaatctatccgcctga

SEQ ID NO: 18 - DNA, 23 bp, Artificial Sequence
  misc_feature (10)..(10): ribonucleotide
cgaatgtaacagacacggtctca

SEQ ID NO: 19 - DNA, 22 bp, Artificial Sequence
  misc_feature (1)..(1): absence or t
nccaagcagtgagtgtgagaan

SEQ ID NO: 20 - DNA, 22 bp, Artificial Sequence
  misc_feature (1)..(2): absence or t
nngacagcgtgaaatcaggnnn

SEQ ID NO: 21 - DNA, 22 bp, Artificial Sequence
  misc_feature (8)..(9): one or both of nucleotides at positions 8 and 9 are ribonucleotide
tgcgaagcatgagctgtgatgg

SEQ ID NO: 22 - DNA, 21 bp, Artificial Sequence
  misc_feature (11)..(14): At least one of nucleotides at positions 11, 12, 13, and 14 are ribonucleotide
ccatcacagcucaugcttcgc

(19) United States
(12) Patent Application Publication
(10) Pub. No.: US 2012/0052492 A1
(43) Pub. Date: Mar. 1, 2012

(54) OLIGONUCLEOTIDES FOR DETECTING LISTERIA SPP. AND USE THEREOF

(75) Inventor: Jun LI, Baltimore, MD (US)

(73) Assignee: SAMSUNG TECHWIN CO., LTD., Changwon-city (KR)

(21) Appl. No.: 13/103,337

(22) Filed: May 9, 2011

Related U.S. Application Data
(60) Provisional application No. 61/378,072, filed on Aug. 30, 2010.

(51) Int. Cl. C12Q 1/68

(52) U.S. Cl. 435/6.11; 435/6.12

(57) ABSTRACT

An oligonucleotide specifically binding to 23S rRNA gene of Listeria spp., and a kit and a method of efficiently detecting Listeria spp. in a sample by using the oligonucleotide are provided.

### CROSS-REFERENCE TO RELATED APPLICATIONS

This application claims benefits from U.S. Provisional Patent Application No. 61/378,072, filed on Aug. 30, 2010, the content of which is hereby incorporated by reference in its entirety.

### FIELD

An oligonucleotide set and a kit for detecting Listeria spp. and a method of detecting Listeria spp. in a sample by using the same are disclosed.

### RELATED ART

Listeria spp. bacteria are gram-positive, non-spore forming and motile bacilli and can grow in a wide temperature range of about −4° C. to about 45° C. and a wide pH range of about ≦5.5 to about 9.5. The Listeria genus contains six species, including Listeria monocytogenes, L. innocua, L. welshimeri, L. seeligeri, L. ivanovii, and L. grayi. Among these species of Listeria, L. monocytogenes is the cause of most human listeriosis cases. The immunocompromised, pregnant women, elderly, and neonates are susceptible to infection caused by this species. Typical symptoms of listeriosis include septicemia, meningitis and miscarriage.

Consumption of contaminated foods is the major cause of Listeria infection. There have been epidemics of various Listeria-induced infections caused by the consumption of contaminated foods, such as unpasteurized milk, contaminated cheese, coleslaw, and the like. Therefore, there is an increasing demand for a method of rapid, sensitive, and accurate detection of Listeria in a sample, such as in a food, a surface wipe, or medical sample.

### SUMMARY

A composition, which is suitable for a rapid, sensitive and accurate detection of Listeria spp. is disclosed. The composition includes a first oligonucleotide of the sequence of SEQ ID NO: 19: X₁CCAAGCAGTGAGTGTGAGAAX₂ (SEQ ID NO:19), wherein X₁ at position 1 is absence or T, and X₂ at position 22 is absence or G, and a second oligonucleotide of the sequence of SEQ ID NO: 20: X₁X₁GACAGCGTGAAATCAGGX₃X₃X₄ (SEQ ID NO: 20), wherein X₁s at positions 1 and 2 are each absence or T; X₃ at position 20 and 21 are absence or A; and X₄ at position 22 is absence or C.

In one embodiment, the number of nucleotide residues in the first oligonucleotide of SEQ ID NO: 19 may be 20 or 21, and the number of nucleotide residues in the second oligonucleotide of SEQ ID NO: 20 is 18-21.

In another embodiment, the first oligonucleotide is one or more selected from the group of oligonucleotides of SEQ ID NOs: 1-3: CCAAGCAGTGAGTGTGAGAAG (SEQ ID NO:1), CCAAGCAGTGAGTGTGAGAA (SEQ ID NO:2), and TCCAAGCAGTGAGTGTGAGAA (SEQ ID NO:3).

In an embodiment, the second oligonucleotide is one or more selected from the group of oligonucleotides of SEQ ID NOs: 5-9: TGACAGCGTGAAATCAGGAAC (SEQ ID NO: 5), TTGACAGCGTGAAATCAGG (SEQ ID NO: 6), TGACAGCGTGAAATCAGGA (SEQ ID NO: 7), TGACAGCGTGAAATCAGGA (SEQ ID NO: 8) and GACAGCGTGAAATCAGGA (SEQ ID NO: 9).

According an embodiment, the composition may further contain a probe oligonucleotide of SEQ ID NO: 21 or SEQ ID NO: 22: TGAGCTGrUrGATGG (SEQ ID NO: 21), wherein at least one of “rU” and “rG” at positions 8 and 9, respectively, are a ribonucleotide, and CCATCACAGCrUrCrArUGCTTCGC (SEQ ID NO. 22), wherein at least one of “rU,” “rC,” “rA,” and “rU” at positions 11, 12, 13, and 14, respectively, is a ribonucleotide. In one embodiment, the probe oligonucleotide has a DNA sequence and an RNA sequence, and is one or more selected from the group consisting of oligonucleotides of SEQ ID NOs: 10-14: TGCGAAGCrATGAGCTGTGATGG (SEQ ID NO: 10), wherein “rA” at position 9 is a ribonucleotide, TGCGAAGrCATGAGCTGTGATGG (SEQ ID NO: 11), wherein “rC” at position 8 is a ribonucleotide, CCATCACAGCTCArUGCTTCGC (SEQ ID NO: 12), wherein “rU” at position 14 is a ribonucleotide, CCATCACAGCTrCrArUGCTTCGC (SEQ ID NO: 13), wherein “rC,” “rA,” and “rU” at positions 12, 13, and 14, respectively, are a ribonucleotide; and CCATCACAGCrUrCrArUGCTTCGC (SEQ ID NO: 14), wherein “rU,” “rC,” “rA,” and “rU” at positions 11, 12, 13, and 14, respectively, are a ribonucleotide. The probe oligonucleotide is labeled with a detectable marker, for example, a fluorescence resonance energy transfer (FRET) pair.

In still another embodiment, a kit for detecting Listeria spp. in a sample, the kit containing the above composition is provided. The kit may further include an amplifying activity and an RNase H. In an embodiment, the kit may further comprise a reverse transcriptase activity for reverse transcription of a target Listeria spp. RNA sequence.

In another embodiment, a method of detecting Listeria spp. in a sample is provided. The method includes (a) amplifying a target nucleic acid of Listeria spp. in the sample to produce an increased number of copies of the target nucleic acid, the amplification including hybridizing a first primer of SEQ ID NO: 19 and a second primer of SEQ ID NO: 20 to the target nucleic acid in the sample to obtain a hybridized product of the target nucleic acid and the primers, and extending the first and the second primers of the hybridized product using a template-dependent nucleic acid polymerase to produce an extended primer product; (b) hybridizing the target nucleic acid to at least one probe oligonucleotide which is capable of being hybridized to the target nucleic acid to obtain a hybridized product of the target nucleic acid:probe oligonucleotide, said probe comprising a DNA sequence and an RNA sequence, and being coupled to a detectable marker; (c) contacting the hybridized product of the target nucleic acid:probe with an RNase H to cleave the probe, resulting in probe fragment dissociation from the target nucleic acid; and (d) detecting the detectable marker. The probe oligonucleotide may be the oligonucleotide of SEQ ID NOs: 21 or 22. The probe oligonucleotide may be one of oligonucleotides of SEQ ID NOs: 10-14. The probe oligonucleotide may be labeled with a detectable marker, for example a fluorescence resonance energy transfer pair.

In another embodiment, a method of detecting a target RNA sequence of Listeria spp. in a sample is provided. The method includes (a) reverse transcribing the Listeria spp. target RNA in the presence of a reverse transcriptase activity and the reverse amplification primer to produce a target cDNA of the target RNA; (b) amplifying the target cDNA sequence to produce an increased number of copies of the target nucleic acid, the amplification including hybridizing a first primer of SEQ ID NO: 19 and a second primer of SEQ ID NO: 20 to the target cDNA to obtain a hybridized product of the target nucleic acid and the primers, and extending the first and the second primers of the hybridized product using a template-dependent nucleic acid polymerase to produce an extended primer product; (c) hybridizing the target nucleic acid to at least one probe oligonucleotide which is substantially complimentary to the target cDNA to obtain a hybridized product of the target nucleic acid:probe oligonucleotide, wherein the probe contains a DNA sequence and an RNA sequence and is coupled to a detectable marker; (d) contacting the hybridized product of the target nucleic acid:probe oligonucleotide with an RNase H to cleave the probe; and (e) detecting an increase in the emission of a signal from the detectable marker on the probe, wherein the increase in signal indicates the presence of the Listeria spp. target RNA in the sample.

Amplification of a target sequence in a sample may be performed by using any nucleic acid amplification method, such as the Polymerase Chain Reaction (U.S. Pat. Nos. 4,683,195, 4,683,202, and 4,800,159) or by using amplification reactions such as Ligase Chain Reaction (Proc. Natl. Acad. Sci. USA 88:189-193), Self-Sustained Sequence Replication (Guatelli et al., 1990, Proc. Natl. Acad. Sci. USA 87:1874-1878), Strand Displacement Amplification (U.S. Pat. Nos. 5,270,184, en 5,455,166), Transcriptional Amplification System (Kwoh et al., Proc. Natl. Acad. Sci. USA 86:1173-1177), Q-Beta Replicase (Lizardi et al., 1988, Bio/Technology 6:1197), Nucleic Acid Sequence Based Amplification (NASBA), Cleavage Fragment Length Polymorphism (U.S. Pat. No. 5,719,028), Isothermal and Chimeric Primer-initiated Amplification of Nucleic Acid (U.S. Pat. No. 6,951,722), Ramification-extension Amplification Method (U.S. Pat. Nos. 5,719,028 and 5,942,391) or other suitable methods for amplification of nucleic acid.

The amplification, hybridization, and contacting steps may be performed simultaneously or sequentially.

In an embodiment, the sample containing Listeria spp. may be cultured in an enrichment medium before the amplification, to enhance growth of the Listeria spp. Such enrichment medium may contain, per 1 L of distilled water, about 10 to about 40 g of tryptic soy broth, about 1 to about 10 g of yeast extract, and about 1 to about 10 g of lithium chloride. The enrichment medium may further contain at least one component selected from the group consisting of about 1 to about 10 g of beef extract, and/or a vitamin mix containing about 0.01 to about 0.5 mg of riboflavin, about 0.5 to about 1.5 mg of thiamine and about 0.01 to about 1.5 mg of biotin; about 1 to about 5 g of pyruvate; and about 0.01 to about 1 g of ferric ammonium citrate. The enrichment medium may further comprise a buffer compound, for example 3-(N-morpholino)propanesulfonic acid (MOPS) and a sodium salt thereof.

In another embodiment, the enrichment medium may contain about 1 to about 10 mg of acriflavine, about 5 to about 15 mg of polymyxin B, and about 10 to about 30 mg of ceftazidime, per 1 L of distilled water. For example, the enrichment medium may contain, per 1 L of distilled water, about 10 to about 40 g of tryptic soy broth, about 1 to about 10 g of yeast extract, about 1 to about 10 g of lithium chloride; about 1 to about 10 g of beef extract and/or a vitamin mix containing about 0.01 to about 0.5 mg of riboflavin, about 0.5 to about 1.5 mg of thiamine, and about 0.01 to about 1.5 mg of biotin; about 1 to about 5 g of pyruvate or a salt thereof; about 0.1 to about 1 g of ferric ammonium citrate; about 4 g of 3-(N-morpholino)propanesulfonic acid (MOPS) and about 7.1 g of sodium MOPS; and about 1 to about 10 mg of acriflavine, about 5 to about 15 mg of polymyxin B, and about 10 to about 30 mg of ceftazidime. In an embodiment, the enrichment medium does not contain one of esculin and peptone, or both.

In still another embodiment, the enrichment medium may contain, per 1 L of distilled water, about 30 g of tryptic soy broth, about 6 g of yeast extract, about 1 to about 10 g of lithium chloride; about 5 g of beef extract and/or a vitamin mix containing about 0.1 mg of riboflavin, about 1.0 mg of thiamine, and about 1.0 mg of biotin; about 2 g of sodium pyruvate; about 0.2 g of ferric ammonium citrate; about 4 g of 3-(N-morpholino)propanesulfonic acid (MOPS) and about 7.1 g of sodium MOPS; and about 5 mg of acriflavine, about 10 mg of polymyxin B, and about 20 mg of ceftazidime.

In another embodiment, the enrichment medium may be brain-heart infusion broth or tryptic soy broth containing 0.6% yeast extract.

The sample may be a food sample, a medical sample, or a surface wipe.

### BRIEF DESCRIPTION OF THE DRAWINGS

FIG. 1 is a graph illustrating the real-time polymerase chain reaction (PCR) results with respect to concentration of Listeria spp. nucleic acid;

FIG. 2 is a graph illustrating the correlation of Cp values of real-time PCR amplification products with the concentration of Listeria spp. nucleic acid;

FIG. 3 is a graph of the real-time PCR amplification results with respect to concentration of an internal amplification control (IAC) target nucleic acid;

FIG. 4 is a graph illustrating the correlation of Cp values of real-time PCR amplification products with the concentration of the IAC target nucleic acid;

FIG. 5 is a graph illustrating the real-time PCR results (inclusivity test) on 92 strains of Listeria. species;

FIG. 6 is a graph illustrating the real-time PCR results on (exclusivity test) 23 non-Listeria species;

FIG. 7 is a graph illustrating the correlation of real-time PCR products with the number of cells of L. monocytogenes;

FIG. 8 is a graph illustrating the results of amplifying Listeria spp. 23S rRNA by one-step RT-PCR using RNase H in different buffers;

FIG. 9 is a graph illustrating the results of RT-PCR performed using Tfi buffer and AgPath buffer; and

FIGS. 10(A)-10(C) show the increase in sensitivity of detection of target RNA when the sample is enriched by culturing it prior to RT-PCR.

### DETAILED DESCRIPTION

The practice of the embodiments described herein employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

The term “amplification” used herein refers to any process for increasing the number of copies of nucleotide sequences. Nucleic acid amplification describes a process whereby nucleotides are incorporated into nucleic acids, for example, DNA or RNA.

The term “nucleotide” used herein refers to a base-sugar-phosphate combination. Nucleotides are the monomeric units of nucleic acids, for example, DNA or RNA. The term “nucleotide” includes ribonucleoside triphosphates, such as rATP, rCTP, rGTP, or rUTP, and deoxy-ribonucleotide triphosphates, such as dATP, dCTP, dGTP, or dTTP.

The term “nucleoside” used herein refers to a base-sugar combination, i.e., a nucleotide lacking phosphate moieties. The terms “nucleoside” and “nucleotide” are used interchangeably in the field. For example, the nucleotide deoxyuridine, dUTP, is a deoxynucleoside triphosphate. It serves as a DNA monomer, for example, being dUMP or deoxyuridine monophosphate, after being inserted into DNA. In this regard, even though no dUTP moiety is present in the result DNA, dUTP may be considered as having been inserted.

The term “polymerase chain reaction (PCR)” generally refers to an amplification method for increasing the number of copies of target nucleic acid(s) in a sample. The procedure is described in detail in U.S. Pat. Nos. 4,683,202, 4,683,195, 4,800,159, and 4,965,188, the contents of which are incorporated herein in their entirety. The sample may include a single nucleic acid or multiple nucleic acids. In general, PCR involves incorporating at least two extendible primer nucleic acids into a reaction mixture containing target nucleic acid(s). The primers are complementary to opposite strands of a double-stranded target sequence. The reaction mixture is subjected to thermal cycling in the presence of a nucleic acid polymerase and nucleic acid monomers, for example, in the presence of dNTP's and/or rNTP's, to amplify the target nucleic acid by extension of the primers. In general, the thermal cycling may involve: annealing to hybridize the primer and target nucleic acid; extending the primers using a nucleic acid polymerase; and denaturating the hybridized primer extension product and the target nucleic acid. The term “reverse transcriptase-PCR (RT-PCR)” is a PCR that uses an RNA template and a reverse transcriptase, or an enzyme having reverse transcriptase activity, to first generate a single stranded cDNA molecule prior to the multiple cycles of DNA-dependent DNA polymerase primer extension. The term “multiplex PCR” refers to PCRs that produce more than two amplified target products in a single reaction, typically by the inclusion of more than two primers.

The term “nucleic acid” used herein refers to a polymer including more than two nucleotides. The term “nucleic acid” is used interchangeably with “polynucleotide” or “oligonucleotide”. Nucleic acids include DNA and RNA. The structure of nucleic acids may be double-stranded and/or single-stranded.

The term “nucleic acid analog” used herein refers to a nucleic acid that contains at least one nucleotide analog and/or at least one phosphate ester analog and/or at least one pentose sugar analog. Examples of nucleic acid analogues include nucleic acids in which the phosphate ester and/or sugar phosphate ester linkages are replaced with other types of linkages, such as N-(2-aminoethyl)-glycine amides and other amides. Nucleic acid analogs refer to a nucleic acid that contains at least one nucleotide analog and/or at least one phosphate ester analog and/or at least one pentose sugar analog and may form a double helix by hybridization.

The terms “annealing” and “hybridization” used herein are interchangeable and refer to the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

The term “probe” used herein refers to a nucleic acid having a sequence complementary to a target nucleic acid sequence and capable of hybridizing to the target nucleic acid to form a duplex. The sequence of the probe may be fully or completely complementary to the target nucleic acid sequence. The probe may be labeled so that the target nucleic acid may be detected simultaneously with PCR.

The terms “target nucleic acid” or “target sequence” used herein includes a full length or a fragment of a target nucleic acid that may be amplified and/or detected. A target nucleic acid may be present between two primers that are used for amplification.

The term “hybrid oligonucleotide” used herein with regard to an oligonucleotide means an oligonucleotide molecule which contains a DNA and an RNA portion within a single molecule. The hybrid oligonucleotide may contain more than one DNA portion and one RNA portion, for example a DNA-RNA, RNA-DNA, or DNA-RNA-DNA oligonucleotide.

In embodiments, an oligonucleotide set for detecting Listeria spp. includes at least one first primer selected from the group consisting of SEQ ID NOs. 1-3; at least one second primer selected from the group consisting of SEQ ID NOs. 5-9; and at least one probe selected from the group consisting of oligonucleotides of SEQ ID NOs. 10-14.

A primer pair containing at least one first primer selected from SEQ ID NOs. 1-3 and at least one second primer selected from SEQ ID NOs. 5-9 have sequences complementary to the respective opposite strands of a target nucleic acid, and may define the target nucleic acid. The primer pair is complementary to the 23S rRNA gene of Listeria spp., and may be used to specifically amplify the target nucleic acid in the 23S rRNA gene. The 23S rRNA gene may be about 3000 bp in length. When used for amplification, the primer pair can amplify target nucleic acid sequences of any Listeria species of the Listeria genus, but not the target nucleic acid sequences of non-Listeria spp. Thus, the primer pair specifically amplifies target nucleic acids of Listeria spp. with single copy sensitivity.

In one embodiment, the probe may have a DNA-RNA-DNA hybrid structure. The probe may be a nucleic acid or a nucleic acid analog. The probe also may be a protected nucleic acid. For example, a DNA or RNA portion of the probe may be partially methylated to be resistant to degradation by an RNA-specific enzyme, for example, an RNase H.

The probe may be modified. For example, the base portion of the probe may be partially or fully methylated. Such modifications may inhibit enzymatic or chemical degradation. The 5′ end or 3′ end —OH group of the nucleic acid probe may be blocked. The 3′ end OH group of the nucleic acid probe may be blocked, thus being rendered incapable of extension by a template-dependant nucleic acid polymerase.

The probe may have a detectable label. The detectable label may be any chemical moiety detectable by any method known in the field. Examples of detectable labels include any moiety detectable by spectroscopy, photochemistry, or by biochemical, immunochemical or chemical means. A suitable method of labeling the nucleic acid probe may be selected according to the type of the label and the positions of the label and probe. Examples of labels include enzymes, enzyme substrates, radioactive substance, fluorescent dyes, chromophores, chemiluminescent labels, electrochemical luminescent label, ligands having specific binding partners, and other labels that interact with each other to increase, vary or reduce the intensity of a detection signal. These labels are durable throughout the thermal cycling for PCR.

The detectable label may be a fluorescence resonance energy transfer (FRET) pair. The detectable label is a FRET pair including a fluorescent donor and a fluorescent acceptor separated by an appropriate distance, and in which donor fluorescence emission is quenched by the acceptor. However, when the donor-acceptor pair is dissociated by cleavage, donor fluorescence emission is enhanced. A donor chromophore, in its excited state, may transfer energy to an acceptor chromophore when the pair is in close proximity This transfer is always non-radiative and occurs through dipole-dipole coupling. Any process that sufficiently increases the distance between the chromophores will decrease FRET efficiency such that the donor chromophore emission can be detected radiatively. Examples of donor chromophores include FAM, TAMRA, VIC, JOE, Cy3, Cy5, and Texas Red. Acceptor chromophores are chosen so that their excitation spectra overlap with the emission spectrum of the donor. An example of such a pair is FAM-TAMRA. In addition, an example of the detectable label is a non-fluorescent acceptor that will quench a wide range of donors. Other examples of appropriate donor-acceptor FRET pairs will be known to those of skill in the art.

In an embodiment, the oligonucleotide probe may be present as a soluble form or free form in a solution. In one embodiment, the oligonucleotide probe can be attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include polystyrene, avidin coated polystyrene beads cellulose, nylon, acrylamide gel and activated dextran, controlled pore glass (CPG), glass plates and highly cross-linked polystyrene. These solid supports are preferred for hybridization and diagnostic studies because of their chemical stability, ease of functionalization and well defined surface area. Solid supports such as controlled pore glass (500 Å, 1000 Å) and non-swelling high cross-linked polystyrene (1000 Å) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to separate the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and is completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the hybridization probe is immobilized on a solid support. The oligonucleotide probe is contacted with a sample of nucleic acids under conditions favorable for hybridization. In an unhybridized state, the fluorescent label is quenched by the acceptor. Upon hybridization to the target, the fluorescent label is separated from the quencher and the fluorescence emission is enhanced.

Immobilization of the hybridization probe to the solid support also enables the target sequence hybridized to the probe to be readily isolated from the sample. In later steps, the isolated target sequence may be separated from the solid support and processed (e.g., purified, amplified) according to methods well known in the art depending on the particular needs of the researcher.

In an embodiment, the oligonucleoride set suitable for detecting Listeria spp. may include a primer of SEQ ID NO. 3; a primer of SEQ ID NO. 7; and a probe of SEQ ID NO. 12.

The oligonucleotide set may be used for amplification and detection of target nucleic acids. The amplification may include extending the primers using a template-dependent polymerase, which results in the formation of PCR fragment or amplicon. The amplification can be accomplished by any method selected from the group consisting of Polymerase Chain Reaction or by using amplification reactions such as Ligase Chain Reaction, Self-Sustained Sequence Replication, Strand Displacement Amplification, Transcriptional Amplification System, Q-Beta Replicase, Nucleic Acid Sequence Based Amplification (NASBA), Cleavage Fragment Length Polymorphism, Isothermal and Chimeric Primer-initiated Amplification of Nucleic Acid, Ramification-extension Amplification Method or other suitable methods for amplification of nucleic acid. The amplification may include simultaneous real-time detection of target nucleic acids

The term “PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment can be single-stranded or double-stranded, or a mixture thereof in any concentration ratio. A PCR fragment can be 100-500 nucleotides or more in length.

An amplification “buffer” is a compound added to an amplification reaction which modifies the stability and/or activity of one or more components of the amplification reaction by regulating the amplification reaction. The buffering agents of the invention are compatible with PCR amplification and RNase H cleavage activity. Examples of buffers include, but are not limited to, HEPES ((4-(2-hydroxyethyl)-1-piperazineethanesulfonic acid), MOPS (3-(N-morpholino)-propanesulfonic acid), and acetate or phosphate containing buffers and the like. In addition, PCR buffers may generally contain up to about 70 mM KCl and about 1.5 mM or higher MgCl₂, and about 50-200 μM each of dATP, dCTP, dGTP and dTTP. The buffers of the invention may contain additives to optimize efficient reverse transcriptase-PCR or PCR reactions.

An additive is a compound added to a composition which modifies the stability and/or activity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)_{2,} LiCl, MnOAc, NMP, trehalose, demiethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll, aurintricarboxylic acid, Tween 20, Tween 21, Tween 40, Tween 60, Tween 85, Brij 30, NP-40, Triton X-100, CHAPS, CHAPSO, Mackemium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. Additives may be optionally added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, a “thermostable polymerase” is an enzyme that is relatively stable to heat and eliminates the need to add enzyme prior to each PCR cycle. Non-limiting examples of thermostable polymerases may include polymerases isolated from the thermophilic bacteria Thermus aquaticus (Taq polymerase), Thermus thermophilus (Tth polymerase), Thermococcus litoralis (Tli or VENT polymerase), Pyrococcus furiosus (Pfu or DEEPVENT polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME polymerase) Thermotoga neapolitana (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include AmpliTaq, AmpliTaq Stoffel fragment, SuperTaq, SuperTaq plus, LA Taq, LApro Taq, and EX Taq.

One of the most widely used techniques to study gene expression exploits first-strand cDNA for mRNA sequence(s) as template for amplification by the PCR. This method, often referred to as reverse transcriptase - PCR, exploits the high sensitivity and specificity of the PCR process and is widely used for detection and quantification of RNA.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” reverse transcriptase PCR methods use a common buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV reverse transcriptase and Taq DNA Polymerase wherein the initial 65° C. RNA denaturation step was omitted.

The first step in real-time, reverse-transcription PCR is to generate the complementary DNA strand using one of the template specific DNA primers. In traditional PCR reactions this product is denatured, the second template specific primer binds to the cDNA, and is extended to form duplex DNA. This product is amplified in subsequent rounds of temperature cycling. To maintain the highest sensitivity it is important that the RNA not be degraded prior to synthesis of cDNA. The presence of RNase H in the reaction buffer will cause unwanted degradation of the RNA:DNA hybrid formed in the first step of the process because it can serve as a substrate for the enzyme. There are two major methods to combat this issue. One is to physically separate the RNase H from the rest of the reverse-transcription reaction using a barrier such as wax that will melt during the initial high temperature DNA denaturation step. A second method is to modify the RNase H such that it is inactive at the reverse-transcription temperature, typically 45-55° C. Several methods are known in the art, including reaction of RNase H with an antibody, or reversible chemical modification. For example, a hot start RNase H activity as used herein can be an RNase H with a reversible chemical modification produced after reaction of the RNase H with cis-aconitic anhydride under alkaline conditions. When the modified enzyme is used in a reaction with a Tris based buffer and the temperature is raised to 95° C. the pH of the solution drops and RNase H activity is restored. This method allows for the inclusion of RNase H in the reaction mixture prior to the initiation of reverse transcription.

Additional examples of RNase H enzymes and hot start RNase H enzymes that can be employed in the invention are described in U.S. Patent Application No. 2009/0325169 to Walder et al., the content of which is incorporated herein in its entirety.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by real-time detection in combination with these reverse transcriptase-PCR techniques has enabled accurate and precise determination of RNA copy number with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“Taq-Man”) or endonuclease assay (sometimes referred to as, “CataCleave”), discussed below.

Post-amplification amplicon detection is both laborious and time consuming Real-time methods have been developed to monitor amplification during the PCR process. These methods typically employ fluorescently labeled probes that bind to the newly synthesized DNA or dyes whose fluorescence emission is increased when intercalated into double stranded DNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red. Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pat. No. 5,925,517), TaqMan probes (e.g., U.S. Pat. Nos. 5,210,015 and 5,487,972), and CataCleave probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded, the distance between the donor and acceptor chromophores increases such that FRET is reversed and donor emission can be monitored using specialized instrumentation. TaqMan and CataCleave technologies differ from the molecular beacon in that the FRET probes employed are cleaved such that the donor and acceptor chromophores become sufficiently separated to reverse FRET.

TaqMan technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan probe binds to one strand of the amplicon at the same time that the primer binds. As the DNA polymerase extends the primer the polymerase will eventually encounter the bound TaqMan probe. At this time the exonuclease activity of the polymerase will sequentially degrade the TaqMan probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan works a specific amplicon can be detected only once for every cycle of PCR. Extension of the primer through the TaqMan target site generates a double stranded product that prevents further binding of TaqMan probes until the amplicon is denatured in the next PCR cycle.

U.S. Pat. No. 5,763,181, the content of which is incorporated herein by reference, describes another real-time detection method (referred to as “CataCleave”). CataCleave technology differs from TaqMan in that cleavage of the probe is accomplished by a second enzyme that does not have polymerase activity. The CataCleave probe has a sequence within the molecule which is a target of an endonuclease, such as a restriction enzyme or RNase. In one example, the CataCleave probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan probe and donor emission can be monitored. Cleavage and dissociation regenerates a site for further CataCleave binding. In this way it is possible for a single amplicon to serve as a target or multiple rounds of probe cleavage until the primer is extended through the CataCleave probe binding site.

In embodiments, the probe used in the method is a CataCleave probe. Examples of suitable CataCleave probes include oligonucleotides comprising the sequence of one of SEQ ID NOS: 21, 22, 10, 11, 12, 13, and 14.

In embodiments, a kit for detecting Listeria spp. in a sample includes the oligonucleotides described above.

The kit may further include a reagent for nucleic acid amplification. The reagent may further include at least one selected from the group consisting of dNTP's, rNTP's, a nucleic acid polymerase, a uracil N-glycosylase (UNG) enzyme, a buffer, and a cofactor (for example, Mg²⁺ ). The nucleic acid polymerase may be selected from the group consisting of a DNA polymerase, a RNA polymerase, and a reverse transcriptase. The nucleic acid polymerase may be thermostable. The nucleic acid polymerase may retain its activity at elevated temperatures, for example, at 95° C. or higher. Thermostable DNA polymerases may be isolated from heat-resistant bacteria selected from the group consisting of Thermus aquaticus, Thermus flavus, Thermus ruber, Thermus thermophilus, Bacillus stearothermophilus, Thermus lacteus, Thermus rubens, Thermotoga maritima, Thermococcus littoralis, and Methanothermus fervidus. An example of a thermostable DNA polymerase is a Taq polymerase. The Taq polymerase is known to have optimal activity at about 70° C.

When the probe is hybridized to a target DNA, the Listeria spp. detection kit may further include a factor specifically cleaving the RNA portion of the DNA-RNA hybrid. The cleaving factor may be RNase H. The cleaving factor may cleave specifically or nonspecifically the RNA portion. A specific RNA cleaving factor may be RNase HI. A nonspecific RNA cleaving factor may be RNase HII. RNase H may hydrolyze RNA in the RNA-DNA hybrid. For RNase H activity, a divalent ion (for example, Mg²⁺, Mn²⁺) is required. The RNase H cleaves RNA 3′-O—P linkages to produce 3′-hydroxyl and 5′-phosphate end products. The RNase H may be selected from the group consisting of a Pyrococcus furiosus RNase HII, a Pyrococcus horikoshi RNase HII, a Thermococcus litoralis RNase HI, and a Thermus thermophilus RNase HI. The Pyrococcus furiosus RNase HII may have an amino acid sequence of SEQ ID NO. 15. The RNase H may be thermostable. For example, the RNase H may retain its activity during a denaturation process in PCR. The cleaving factor may be a reversibly modified form of a thermostable RNase HII, which is inactive in its modified form and active in its unmodified form, wherein the modification is a coupling of the RNase HII to a ligand, crosslinking of the RNase HII, or chemical reaction of an amino acid residue in the RNase HII, and wherein the enzymatic activity of the modified RNase HII is restored by heating or adjusting pH of a sample containing the RNase HII.

When the RNA portion of the probe that contains a DNA sequence and an RNA sequence is cleaved by the cleaving factor, dissociation may occur. Such dissociation may naturally occur due to a decrease in the melting temperature of the cleaved complex or may be facilitated by a factor, such as temperature elevation. Dissociated fragments may be detected by any method known in the field.

In embodiments, a method of detecting Listeria spp. in a sample includes: (a) amplifying a target nucleic acid of Listeria spp. in the sample to produce an increased number of copies of the target nucleic acid, the amplifying including hybridizing a first primer of SEQ ID NO: 19 and a second primer of SEQ ID NO: 20 to the target nucleic acid in the sample to obtain a hybridized product of the target nucleic acid and the primers, and extending the first and the second primers of the hybridized product using a template-dependent nucleic acid polymerase to produce an extended primer product; (b) hybridizing the target nucleic acid to at least one probe oligonucleotide which is capable of being hybridized to the target nucleic acid to obtain a hybridized product of the target nucleic acid:probe oligonucleotide, wherein the probe contains an RNA sequence and a DNA sequence, and is coupled to a detectable marker; (c) contacting the hybridized product of the target nucleic acid:probe with RNase H to cleave the probes, resulting in probe fragment dissociation from the target nucleic acid; and (d) detecting the detectable marker.

Amplification of a target sequence in a sample may be performed by using any nucleic amplification method, such as the Polymerase Chain Reaction or by using amplification reactions such as Ligase Chain Reaction, Self-Sustained Sequence Replication, Strand Displacement Amplification, Transcriptional Amplification System, Q-Beta Replicase, Nucleic Acid Sequence Based Amplification (NASBA), Cleavage Fragment Length Polymorphism, Isothermal and Chimeric Primer-initiated Amplification of Nucleic Acid, Ramification-extension Amplification Method or other suitable methods for amplification of nucleic acid.

In an embodiment, the method includes amplifying a target nucleic acid fragment of Listeria spp., the amplifying including hybridizing at least one primer selected from SEQ ID NOs. 1-3 and at least one primer selected from SEQ ID NOs. 5-9 to the target nucleic acid in the sample to obtain a hybridized product; and extending the primers of the hybridized product using a template-dependent nucleic acid polymerase to produce an extended primer product; hybridizing the target nucleic acid fragment to at least one probe selected from the group consisting of oligonucleotides of SEQ ID NOs. 10-14 to obtain a hybridized product; contacting the hybridized product from the target nucleic acid fragment and the probe to a RNase H to cleave the probes, resulting in a probe fragment dissociating from the hybridized product; and detecting the detectable marker.

Hereinafter, the method will now be described in greater detail. The method includes amplifying a target nucleic acid fragment of Listeria spp., the amplification including hybridizing at least one primer selected from SEQ ID NOs. 1-3 and at least one primer selected from SEQ ID NOs. 5-9 to the target nucleic acid in the sample to obtain a hybridized product; and extending the primers of the hybridized product depending on a template using a template-dependent nucleic acid polymerase to produced an extended primer product.

The hybridization may be conducted in a liquid medium. A suitable liquid medium may be selected according to the requirement(s). The liquid medium may be, for example, water, a buffer, or a PCR mixture. Nonlimiting examples of buffers include PBS, Tris, MOPS and Tricine. The hybridization may be conducted under the conditions to facilitate the binding of the primer and the target nucleic acid, for example, at low temperatures and low salt concentrations. Those conditions to facilitate hybridization are known in the field. The target nucleic acid may be a single-stranded or double-stranded nucleic acid. For example, a double-stranded target nucleic acid may be denaturated into separate single strands. The target nucleic acid may be DNA or RNA.

The extending of the primer depending on a template refers to polymerization, which is known in the field. The nucleic acid polymerase may be thermostable.

The method of detecting Listeria spp. includes hybridizing the target nucleic acid fragment to at least one probe selected from the group consisting of oligonucleotides of SEQ ID NOs. 10-14 to obtain a hybridized product. The probes as described above may be used. The probe may be labeled with a detectable marker, for example, an optically detectable marker. Detectable markers are known in the art and may be suitably selected. For example, a FRET pair may be used for the purpose of detecting the target sequence in an embodiment of the invention.

The hybridization may be conducted in a liquid medium. A suitable liquid medium may be selected according to the requirement(s). The liquid medium may be, for example, water, a buffer, or a PCR mixture. Nonlimiting examples of buffers include PBS, Tris, MOPS (3-(N-morpholino)propanesulfonic acid) and Tricine. The hybridization may be conducted under the conditions to facilitate the binding of the single-stranded nucleic acid probe and the target nucleic acid, for example, at low temperatures and low salt concentrations. Those conditions to facilitate hybridization are known in the field. The target nucleic acid may be a single-stranded or double-stranded nucleic acid. For example, a double-stranded target nucleic acid may be denaturated into separate single strands, as described above. The target nucleic acid may be DNA or RNA.

The method of detecting Listeria spp. includes contacting the hybridized product from the target nucleic acid fragment and the probe to a RNase H to cleave the probe, resulting in probe fragment dissociating from the hybridized product; and The hybridized product and the RNase H may contact each other in a liquid medium. A suitable liquid medium may be selected according to the requirement(s). The liquid medium may be, for example, water, a buffer, or a PCR mixture. Nonlimiting examples of buffers include PBS, Tris, MOPS (3-(N-morpholino)propanesulfonic acid) and Tricine. The contact may be conducted under substantially the same conditions as PCR conditions or in a PCR mixture.

The RNase H may be RNase HI or RNase HII. The RNase H may hydrolyze RNA in the RNA-DNA hybrid. For RNase H activity, a divalent ion (for example, Mg²⁺, Mn²⁺) is required. The RNase H cleaves RNA 3′-O—P linkages to produce 3′-hydroxyl and 5′-phosphate end products. The RNase H may be selected from the group consisting of a Pyrococcus furiosus RNase HII, a Pyrococcus horikoshi RNase HII, a Thermococcus litoralis RNase HI, and a Thermus thermophilus RNase HI. The Pyrococcus furiosus RNase HII may have an amino acid sequence of SEQ ID NO. 15. The RNase H may be thermostable. For example, the RNase H may retain its activity during a denaturation process in PCR. The RNase H may be a reversibly modified form of a thermostable RNase HII, which is inactive in its modified form and active in its unmodified form, wherein the modification is a coupling of the RNase HII to a ligand, crosslinking of the RNase HII, or chemical modification of the RNase HII, and wherein the enzymatic activity of the modified RNase HII is restored by heating or adjusting the pH of a sample containing the RNase HII.

Such dissociation may naturally occur due to the binding force of the strands that is weaken by the cleavage or may be facilitated by a factor, such as temperature elevation. For example, the PCR mixture may include an RNase H enzyme that will specifically cleave the RNA sequence portion of a RNA-DNA duplex. After cleavage, the two halves of the probe dissociate from a target amplicon at the reaction temperature and diffuse into the reaction buffer. As the donor and acceptors separate, FRET is reversed and donor emission can be monitored. Cleavage and dissociation regenerates a site for further probe binding. In this way it is possible for a single amplicon to serve as a target or multiple rounds of probe cleavage until the primer is extended through the probe binding site.

The method of detecting Listeria spp. includes detecting the probe nucleic acid fragment. The detection of the probe nucleic acid fragment may be carried out by any of a variety of methods, which are appropriately chosen according to the detectable markers. Throughout the specification, the term “a detectable marker” and “a detectable label” are used interchangeably. For example, the size of reaction products may be analyzed to detect the labeled probe fragment. The analysis of the size of the probe nucleic acid fragment may be carried out by any known method, for example, gel electrophoresis, gradient sedimentation, size exclusion chromatography, or homochromatography. When the detectable label used is a FRET pair, the labeled probe fragment may be identified in-situ by spectroscopy, without performing size analysis. Thus, real-time detection of the labeled probe fragment is achievable.

The method of detecting Listeria spp. may further include cultivating the sample containing Listeria spp. species in an enrichment medium before the amplification process, to enhance growth of the Listeria spp. species.

The enrichment medium used for the cultivation may have the following features. The enrichment medium may not contain at least one selected from esculin and peptone. In another embodiment, the enrichment medium may contain esculin as long it does not interfere with any of the steps performed according to the embodiments of the invention, for example amplification of a target sequence or detecting the target sequence by cleaving the labeled probe and detecting the cleaved labeled probe. The enrichment medium may be a medium for enhancing growth of Listeria spp. species, containing, per 1 L of distilled water, about 10 to about 40 g of tryptic soy broth (TSB), about 1 to about 10 g of yeast extract (YE), and about 1 to about 15 g of lithium chloride. The enrichment medium may further contain at least one component selected from the group consisting of about 1 to about 10 g of beef extract (BE), or a vitamin mix containing about 0.01 to about 0.5 mg of riboflavin, about 0.5 to about 1.5 mg of thiamine and about 0.01 to about 1.5 mg of biotin; about 1 to about 5 g of pyruvate or a salt thereof; and about 0.01 to about 1 g of ferric ammonium citrate. The enriched medium may further contain a buffer compound. The buffer compound may include 3-(N-morpholino)propanesulfonic acid (MOPS) free acid and a sodium salt. For example, the enriched medium may contain about 4 g of MOPS free acid and about 7.1 g of sodium MOPS. Alternatively, the enriched medium may contain about 1 to about 10 mg of acriflavine, about 5 to about 15 mg of polymyxin B, about 10 to about 30 mg of ceftazidime, and about 10 to about 60 mg of nalidixic acid.

The enrichment medium may be a medium containing, per 1 L of distilled water, about 10 to about 40 g of tryptic soy broth (TSB), about 1 to about 10 g of yeast extract (YE), about 1 to about 10 g of lithium chloride; about 1 to about 10 g of beef extract (BE) and/or a vitamin mix containing about 0.01 to about 0.5 mg of riboflavin, about 0.5 to about 1.5 mg of thiamine, and about 0.01 to about 1.5 mg of biotin; about 1 to about 5 g of pyruvate or a salt thereof; about 0.01 to 1 g of ferric ammonium citrate; about 4 g of MOPS free acid and about 7.1 g of sodium MOPS; and about 1 to about 10 mg of acriflavine, about 5 to about 15 mg of polymyxin B, and about 10 to about 30 mg of ceftazidime. For example, the enrichment medium may be a medium containing 30 g of tryptic soy broth (TSB), 6 g of yeast extract, 1 g of esculin, 10 g of LiCl, 2 g of sodium pyruvate, 0.1 g of ferric ammonium citrate, 8 g of MOPS free acid, 14.2 g of MOPS, sodium, 5 g of beef extract, and a vitamin mix containing about 0.1 mg of riboflavin, about 1.0 mg of thiamine, and about 1.0 mg of biotin; or a medium (sometimes, referred to as A2.2 medium) containing, per 1 L of distilled water, about 30 g of tryptic soy broth (TSB), about 6 g of yeast extract (YE), about 1 to about 10 g of lithium chloride; 5 g of beef extract (BE) and/or a vitamin mix containing about 0.1 mg of riboflavin, about 1.0 mg of thiamine, and about 1.0 mg of biotin; 2 g of sodium pyruvate; about 0.1 g of ferric ammonium citrate; about 4 g of MOPS free acid and about 7.1 g of sodium MOPS; about 5 mg of acriflavine, about 10 mg of polymyxin B, and about 20 mg of ceftazidime. Using such an enrichment medium may eliminate or reduce PCR inhibitors in culture products and promote growth of Listeria species while inhibiting growth of background microflora, thus enabling efficient detection of Listeria spp. in a sample.

Enrichment medium may be BHI (brain heart infusion) broth, which may be used as it is or supplemented with trace ingredients such as sodium chloride and/or disodium phosphate. BHI is commercially available from different sources, under different tradenames such as BACTO®, BBL®, or Difco®. Enrichment medium may also be tryptic soy broth (TSB) with or without supplement of 0.6% yeast extract.

An exemplary protocol for detecting a target Listeria spp. sequence may include the steps of providing a food sample or surface wipe, mixing the sample or wipe with a growth medium and incubating to increase the number or population of Listeria (“enrichment”), disintegrating Listeria cells (“lysis”), and subjecting the obtained lysate to amplification and detection of target Salmonella sequence. Food samples may include, but are not limited to, fish such as smoked salmon, dairy products such as milk and cheese, and liquid eggs, poultry, fruit juices, meats such as ground pork, pork, ground beef, or beef, or deli meat, vegetables such as spinach, or environmental surfaces such as stainless steel, rubber, plastic, and ceramic.

The limit of detection (LOD) for food contaminants is described in terms of the number of colony forming units (CFU) that can be detected in either 25 grams of solid or 25 mL of liquid food or on a surface of defined area. By definition, a colony-forming unit is a measure of viable bacterial numbers. Unlike indirect microscopic counts where all cells, dead and living, are counted, CFU measures viable cells. One CFU (one bacterial cell) will grow to form a single colony on an agar plate under permissive conditions. The United States Food Testing Inspection Service defines the minimum LOD as 1 CFU/25 grams of solid food or 25 mL of liquid food or 1 CFU/surface area.

In practice, it is impossible to reproducibly inoculate a food sample or surface with a single CFU and insure that the bacterium survives the enrichment process. This problem is overcome by inoculating the sample at either one or several target levels and analyzing the results using a statistical estimate of the contamination called the Most Probable Number (MPN). As an example, a Listeria culture can be grown to a specific cell density by measuring the absorbance in a spectrophotometer. Ten-fold serial dilutions of the target are plated on agar media and the numbers of viable bacteria are counted. This data is used to construct a standard curve that relates CFU/volume plated to cell density. For the MPN to be meaningful, test samples at several inoculum levels are analyzed. After enrichment and extraction a small volume of sample is removed for real-time analysis. The ultimate goal is to achieve a fractional recovery of between 25% and 75% (i.e. between 25% and 75% of the samples test positive in the assay using RT-PCR employing a CataCleave probe, which will be explained below). The reason for choosing these fractional recovery percentages is that they convert to MPN values of between 0.3 CFU and 1.375 CFU for 25 gram samples of solid food, 25 mL samples of liquid food, or a defined area for surfaces. These MPN values bracket the required LOD of 1 CFU/sample. With practice, it is possible to estimate the volume of diluted innoculum (based on the standard curve) to achieve these fractional recoveries.

Various embodiments will be described in further detail with reference to the following examples. These examples are for illustrative purposes only and are not intended to limit the scope of the invention.

### EXAMPLE 1

### Real-Time PCR Amplification of Listeria spp. Using Primer Pair of SEQ ID NOs. 3 and 7 and Probe of SEQ ID NO. 12

A primer pair of SEQ ID NOs. 3 and 7 and a probe of SEQ ID NO. 12 were used to amplify and detect a target nucleic acid of Listeria spp. in a sample according to real-time PCR amplification.

(1) Standard Curve and Detection Limit

The correlation of real-time PCR amplification of Listeria spp. using the primer pair of SEQ ID NOs. 3 and 7 and the probe of SEQ ID NO. 12 with the concentration of Listeria spp. was identified.

Serial 10-fold dilutions, from 10⁴ to 10¹⁰ folds, 10¹³ copies/ml of a plasmid including the 23S rDNA of Listeria monocytogenes with a buffer were used as templates in PCR. PCR was performed in the presence of RNase H to induce cleavage of the probe during the PCR. The resulting probe fragments were measured in real time.

The PCR mixture composition and the PCR conditions are as follows.

TABLE 1
Reaction mixture composition:

| Component | μL per 25 μL reaction |
|---|---|
| 10x ICAN PCR buffer | 2.5 |
| Forward primer (20 μM) | 0.5 |
| Reverse primer (20 μM) | 0.5 |
| CataCleave probe (5 μM) | 1 |
| dN/UTP, 2/4 mM | 1 |
| Taq polymerase (5 units/μL) | 0.5 |
| UNG (10 units/μL) | 0.1 |
| RNaseH II (5 units/μL) | 0.2 |
| DNA template | 2 |
| Water | 16.70 |

In Table 1, 1× ICAN PCR buffer indicates a buffer containing 32 mM HEPES (pH 7.8, titrated by concentrated KOH), 100 mM potassium acetate, 4 mM magnesium acetate, 1% DMSO and 0.11% BSA; Forward primer and Reverse primer indicate primers of SEQ ID NOs. 3 and 7; and CataCleave probe indicates a probe of SEQ ID NO. 12 with the 5′ end labeled with FAM and the 3′ end labeled with Iowa Black FQ (Black Hole Quencher) for short wavelength emission. The purified plasmid as a template DNA was mixed with the primers. Pfu RNase HII indicates an RNA-specific thermostable RNase HII enzyme originated from Pyrococcus furiosus.

TABLE 2
Reaction conditions:

| Step | Temp (° C.) | Time (sec) | Cycles |
|---|---|---|---|
| UNG incubation | 37 | 600 | 1 |
| Taq activation/ | 95 | 600 | 1 |
| UNG inactivation/ | | | |
| Initial denaturation | | | |
| Cycles | 95 | 15 | 50 |
| | 60 | 20 | |

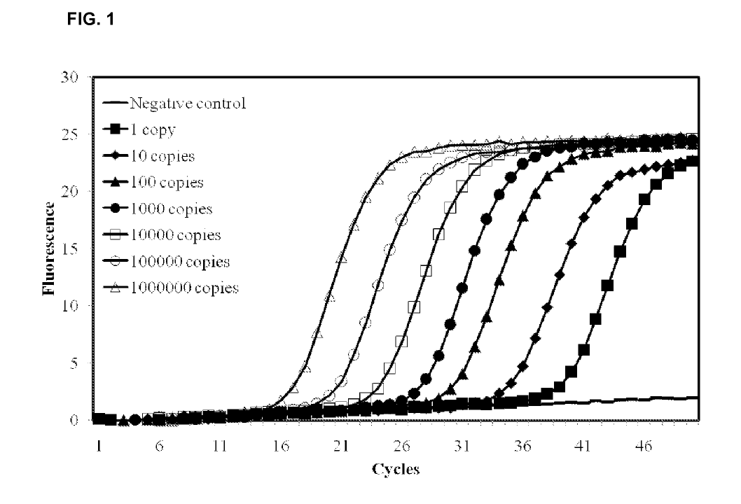

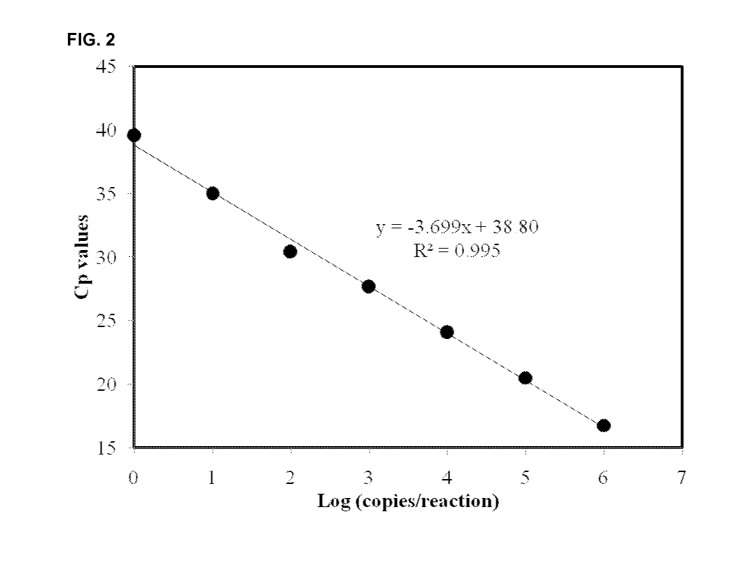

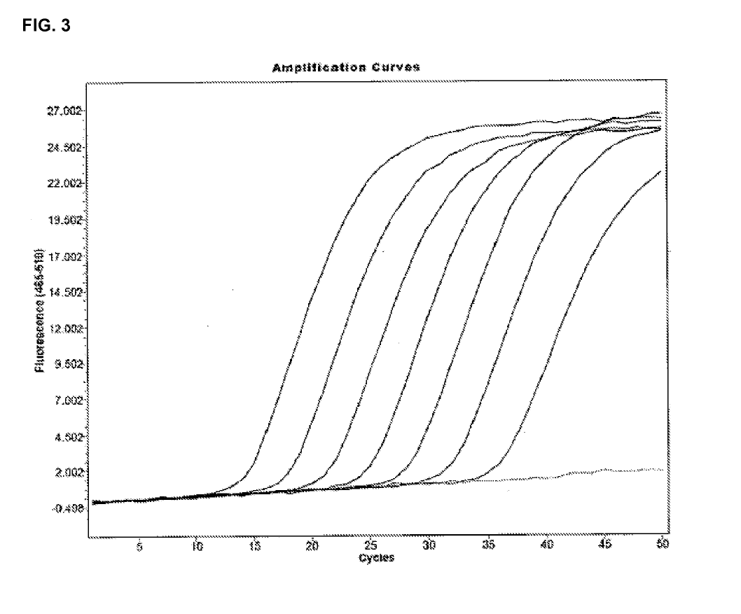

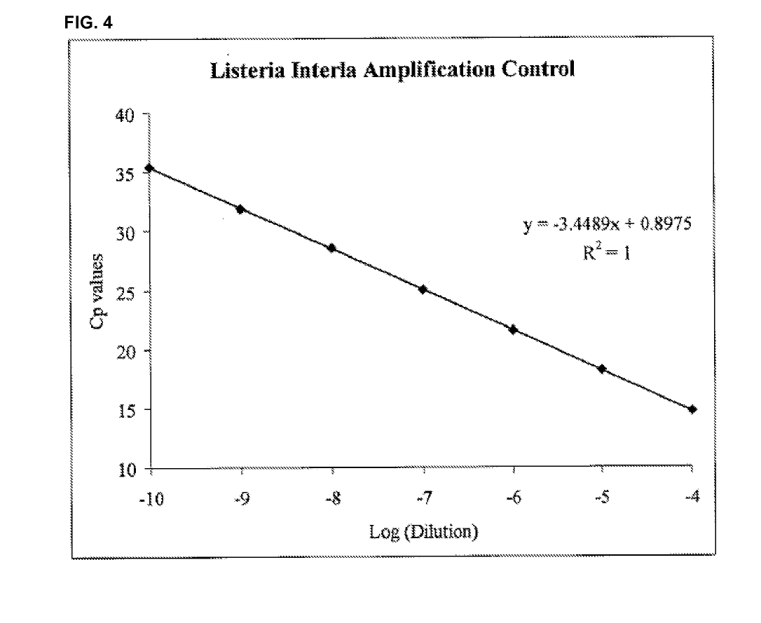

Results are shown in FIGS. 1 and 2. FIG. 1 is a graph illustrating the real-time PCR with primer pair of SEQ ID NOs: 3 and 7 in combination with the Catacleave probe of SEQ ID NO: 12 is able to detect a signal copy of Listeria genomic DNA within 40 or less amplification cycles FIG. 2 is a graph illustrating the correlation of Cp values of real-time PCR amplification products with the concentration of the target nucleic acid.

(2) Inclusivity Test

For the inclusivity test, 92 Listeria strains representing all 6 Listeria species were cultivated overnight in a Brain Heart Infusion medium at 35° C. 5 μL of test cell suspension was extracted in 45 μl of CZ lysis solution (0.3125 mg/ml NaN₃, 12.5 mM Tris (pH 8), 0.25% CHAPS and 1 mg/ml proteinase K) at 55° C. for 15 min followed by 95° C. for 10 mM 2 μL of the resulting lysate was used as template. Table 3 below lists the name of strains tested.

TABLE 3
List of Listeria strains for the inclusivity test

| Listeria species | Serotype | Strain |
|---|---|---|
| Listeria monocytogenes | 1/2c | CDL 36 |
| Listeria monocytogenes | 1/2c | CDL 37 |
| Listeria monocytogenes | 1/2c | CDL 38 |
| Listeria monocytogenes | 3a | CDL 39 |
| Listeria monocytogenes | 3a | CDL 41 |
| Listeria monocytogenes | 3b | CDL 42 |
| Listeria monocytogenes | 3b | CDL 43 |
| Listeria monocytogenes | 3b | CDL 45 |
| Listeria monocytogenes | 3c | CDL 47 |
| Listeria monocytogenes | 3c | CDL 48 |
| Listeria monocytogenes | 3c | CDL 49 |
| Listeria monocytogenes | 3c | CDL 50 |
| Listeria monocytogenes | 4a | CDL 51 |
| Listeria monocytogenes | 4a | CDL 111 |
| Listeria monocytogenes | 1/2b | CDL 112 |
| Listeria monocytogenes | 1/2c | CDL 113 |
| Listeria monocytogenes | 3b | CDL 114 |
| Listeria monocytogenes | 3b | CDL 115 |
| Listeria monocytogenes | 1/2a | CDL 116 |
| Listeria monocytogenes | 4a | CDL 117 |
| Listeria monocytogenes | 4b | CDL 118 |
| Listeria monocytogenes | 4d/e | CDL 120 |
| Listeria monocytogenes | 7 | CDL 122 |
| Listeria monocytogenes | 4b | CDL 123 |
| Listeria monocytogenes | 1/2b | CDL 125 |
| Listeria monocytogenes | 1/2b | CDL 128 |
| Listeria monocytogenes | 1/2b | CDL 131 |
| Listeria monocytogenes | 1/2b | CDL 132 |
| Listeria monocytogenes | 4b | CDL 136 |
| Listeria monocytogenes | 4b | CDL 137 |
| Listeria monocytogenes | 4b | CDL 138 |
| Listeria monocytogenes | 4b | CDL 139 |
| Listeria monocytogenes | 4b | CDL 140 |
| Listeria monocytogenes | 1/2b | CDL 142 |
| Listeria monocytogenes | 1/2b | CDL 143 |
| Listeria monocytogenes | 1/2a | CDL 144 |
| Listeria monocytogenes | 1/2a | CDL 145 |
| Listeria monocytogenes | 1/2b | CDL 147 |
| Listeria monocytogenes | 3b | CDL 149 |
| Listeria monocytogenes | 1/2c | ATCC 19112, CWD 106 |
| Listeria monocytogenes | 4c | ATCC 19116, CWD 108 |
| Listeria monocytogenes | 4b | CWD 1559 |
| Listeria monocytogenes | 3b | CWD 1591 |
| Listeria monocytogenes | 1/2b | CWD 1597 |
| Listeria monocytogenes | 3b | CWD 1600 |
| Listeria monocytogenes | 1/2a | CWD 1609 |
| Listeria monocytogenes | 4b | ATCC 51414, CWD 104 |
| Listeria monocytogenes | 4d | ATCC 19117, CWD 109 |
| Listeria monocytogenes | 4e | ATCC 19118, CWD 110 |
| Listeria monocytogenes | 1/2a | CWD 72 |
| Listeria innocua | 6a | CDL 191 |
| Listeria innocua | 4ab | CDL 192 |
| Listeria innocua | 6b | CDL 236 |
| Listeria innocua | 6a | CDL 237 |
| Listeria innocua | 6a | CDL 240 |
| Listeria innocua | 6b | CDL 241 |
| Listeria innocua | 4ab | CDL 259 |
| Listeria innocua | | L80/20 |
| Listeria innocua | | L80/22 |
| Listeria innocua | | L80/24 |
| Listeria innocua | | L82/14 |
| Listeria innocua | | L82/16 |
| Listeria innocua | | L82/18 |
| Listeria innocua | 6a | DA-20, CWD 181 |
| Listeria welshimeri | 6a | CDL 209 |
| Listeria welshimeri | 6b | CDL 243 |
| Listeria welshimeri | | L82/2, LFD 5121 |
| Listeria welshimeri | | L82/10, LFD 5125 |
| Listeria welshimeri | | L21/44, LFD 782 |
| Listeria welshimeri | | L21/46, LFD 783 |
| Listeria welshimeri | | L21/40, LFD 860 |
| Listeria welshimeri | 6a | ATCC 35897, CWD 114 |
| Listeria seeligeri | 1/2b | CDL 84 |
| Listeria seeligeri | 4c | CDL 98 |
| Listeria seeligeri | 1/2b | ATCC 35967, CWD 166 |
| Listeria ivanovii | 5 | L45/74, LFD 2949 |
| Listeria ivanovii | | L24/6 |
| Listeria ivanovii | | L24/22, LFD 891 |
| Listeria ivanovii | 5 | ATCC 19119, CWD 164 |
| Listeria grayi | | ATCC 25400, CWD 671 |
| Listeria grayi | | ATCC 25401, CWD 673 |
| Listeria grayi | | ATCC 25402, CWD 20 |
| Listeria grayi | | ATCC 25403, CWD 672 |
| Listeria grayi | | ATCC 19120, CWD 2091 |

Real-time PCR was conducted in the presence of a primer pair of SEQ ID NOs. 3 and 7, and a probe of SEQ ID NO. 12. The PCR conditions and the PCR mixture composition were the same as in Tables 1 and 2, respectively.

A total of 92 Listeria species were used in the experiment: 59 strains of L. monocytenges, and 33 strains of other Listeria species including L. innocua, L. ivanovii, L. welshimeri, L. seeligeri, and L. grayi. A PCR mixture containing no template DNA was used as a negative control group.

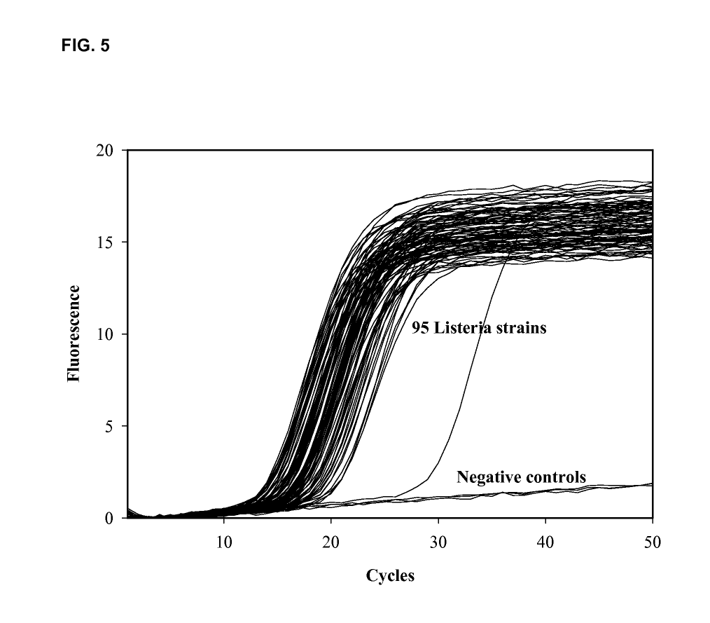

FIG. 5 is a graph illustrating the real-time PCR results on 92 strains of Listeria species. Referring to FIG. 5, only one out of 5 L. grayi strains had a high Cp value, and the rest were efficiently detected in the real-time PCR using the primer pair of SEQ ID NOs. 3 and 7 and the probe of SEQ ID No. 12. This result indicates that real-time PCR assay using the primer pair of SEQ ID NOs. 3 and 7 and the probe of SEQ ID NO. 12 are highly specific to Listeria spp. strains.

(3) Exclusivity Test

For the exclusivity test, 23 non-Listeria species were cultivated to their maximal density in Brain Heart Infusion medium. 5 μL of test cell suspension was extracted in 45 μl of CZ lysis solution (0.3125 mg/ml NaN₃, 12.5 mM Tris (pH 8), 0.25% CHAPS and 1 mg/ml proteinase K) at 55° C. for 15 min followed by 95° C. for 10 min. 2 μL of the resulting lysate was used as template. The PCR conditions and the PCR mixture composition were the same as in Tables 1 and 2, except that 23 non-Listeria species were used.

Twenty-three (23) non-Listeria species used in the experiment include Bacillus mycoides, Brochothrix campestris, Carnobacterium divergens, Carnobacterium malaroma, Enterobacter aerogenes, Enterobacter cancerogenus, Enterobacter cloacae, Enterobacter intermedia, Enterobacter sakazkii, Escherichia coli, Escherichia coli O157:H7, Klebsiella pneumoniae, Kurthia zopfii, Lactococcus lactis, Proteus hauseri, Proteus mirabilis, Proteus vulgaris, Rhodococcus aqui, Staphylococcus aureus, Staphylococcus saprophyticus, Streptococcus agalactiae, Streptococcus dysgalactiae, and Streptococcus sanguinis. A plasmid containing the target gene fragment was used a positive control.

TABLE 4
List of organisms tested for exclusivity test

| | | | Growth |
|---|---|---|---|
| | | | Temperature |
| Organisms | Source (strain) | Origin | (° C.) |
| Bacillus mycoides | ATCC10206 | Unknown | 30 |
| Brochothrix campestris | ATCC43754 | Soil | 26 |
| Carnobacterium divergens | ATCC35677 | Beef | 30 |
| Carnobacterium gallinarum | ATCC49517 | Chicken | 26 |
| Carnobacterium | ATCC43224 | Beef | 26 |
| maltaromaticum | | | |
| Enterobacter aerogenes | ATCC 13048 | Sputum | 37 |
| Enterobacter cancerogenus | ATCC 35317 | Human | 37 |
| Enterobacter cloacae | ATCC 13047 | Spinal | 37 |
| | | Fluid | |
| Enterobacter intermedia | ATCC 33110 | Water | 37 |
| Enterobacter sakazakii | ATCC | Human | 37 |
| | BAA-894 | | |
| Erysipelothrix rhusiopathiae | ATCC19414 | Pig | 37 |
| Escherichia coli | ATCC 11303 | | 37 |
| Escherichia coli O157:H7 | ATCC 43895 | Meat | 37 |
| Klebsiella pneumoniae | ATCC 13883 | | 37 |
| Kurthia zopfii | ATCC33403 | Turkey | 26 |
| Lactobacillus casei | ATCC393 | Cheese | 37 |
| Lactobacillus plantarum | ATCC10012 | Unknown | 37 |
| Lactococcus lactis | ATCC11454 | Unknown | 37 |
| Micrococcus aurantiacus | ATCC11731 | Unknown | 37 |
| Propionibacterium | ATCC13673 | Unknown | 30 |
| freudenreichii | | | |
| Proteus hauseri | ATCC 13315 | | 37 |
| Proteus mirabilis | ATCC 35659 | | 37 |
| Proteus vulgaris | ATCC 33420 | Clinical | 37 |
| | | Isolate | |
| Rhodococcus equi | ATCC10146 | Horse | 37 |
| Staphylococcus aureus | ATCC10832 | Unknown | 37 |
| Staphylococcus epidermidis | ATCC12228 | Unknown | 37 |
| Staphylococcus saprophyticus | ATCC15305 | Urine | 37 |
| Streptococcus agalactiae | ATCC12386 | Unknown | 37 |
| Streptococcus dysgalactiae | ATCC12388 | Human | 37 |
| Streptococcus sanguinis | ATCC10556 | Human | 37 |

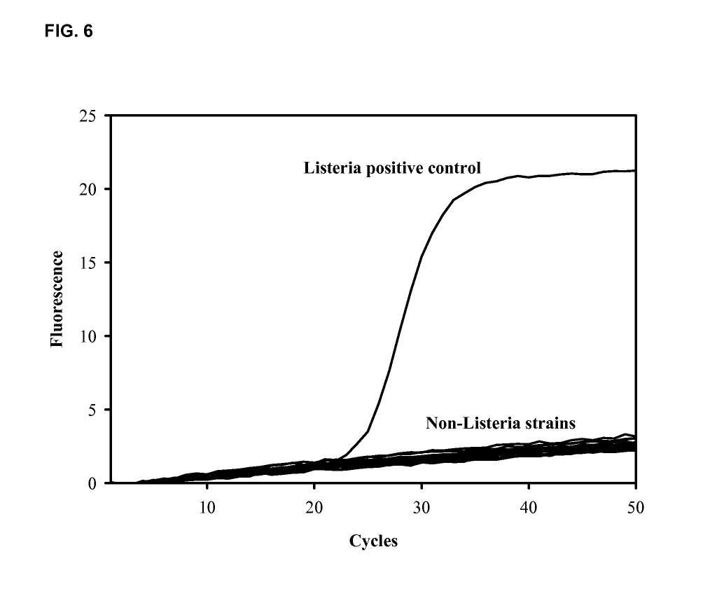

FIG. 6 is as graph illustrating the real-time PCR results on 23 non-Listeria species. Referring to FIG. 6, none of the 23 non-Listeria species were amplified in the real-time PCR using the primer pair of SEQ ID NOS. 3 and 7 and the probe of SEQ ID NO. 12. However, the positive control group was amplified. These results indicate that real-time PCR using the primer pair of SEQ ID NOS. 3 and 7 and the probe of SEQ ID NO. 12 are highly specific to Listeria spp. species.

(4) Detection Limit Test

The correlation of the real-time PCR amplification products with the concentration of cells containing a target DNA was identified.

First, L. monocytogenes was cultivated overnight in a brain heart infusion (BHI) medium at 35° C. The resulting culture products were serially diluted by ten folds in new BHI medium. Each dilution was dissolved in a TZ lysis buffer. The resulting solutions were used in PCR. The cell concentrations were determined using plate counts. PCR amplification was conducted on each Listeria spp. species in the presence of a primer pair of SEQ ID NOS. 3 and 7 and a probe of SEQ ID NO. 12 specific to the 23S rDNA of the Listeria species. The PCR conditions and the PCR mixture composition were the same as in Tables 1 and 2.

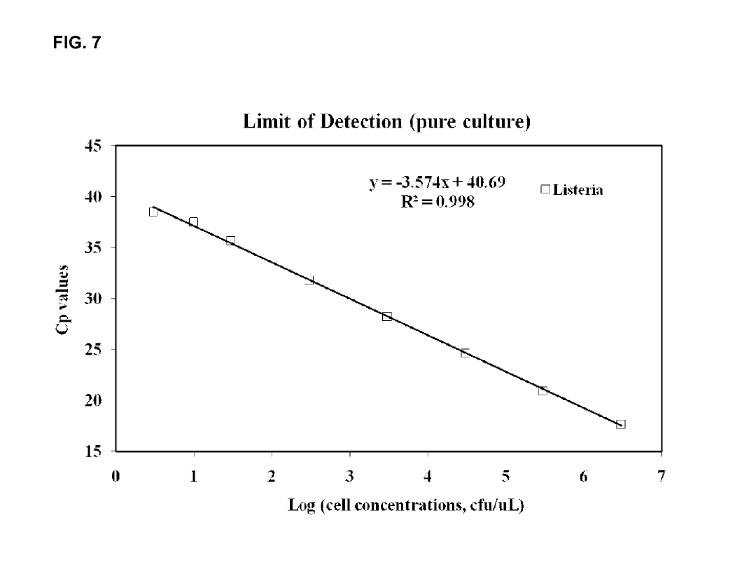

FIG. 7 is a graph illustrating the correlation of real-time PCR products with the number of cells of L. monocytogenes. The detection limit was determined by the normalization of Cp values to the concentration of cells measured using plate counts. As a result, the detection limit (LOD) on Listeria was about 3 cfu/μl.

The results of FIG. 7 indicate that the real-time PCR amplification and detection using the primer pair of SEQ ID NOs. 3 and 7 and the probe of SEQ ID NO. 12 are suitable to detect Listeria spp. species in a sample at a high sensitivity.

### EXAMPLE 2

### Specific Detection of Listeria spp. in Contaminated Sample

A sample contaminated with Listeria spp. was subjected to real-time PCR to amplify and a target nucleic acid of Listeria spp. in the presence of a primer pair of SEQ ID NOs. 3 and 7 and a probe of SEQ ID NO. 12 to detect Listeria spp. in the sample.

(1) Specific Detection of Listeria spp. in Contaminated Liquid Egg and Whole Milk

Liquid egg was inoculated with L. innocua to concentrations of about 1 cfu/25 ml and about 4 cfu/25 ml, respectively. After incubation of the samples at 4° C. for 24 hours, each sample was cultivated in an enriched medium A2.2 (propriety formulation containing 30 g/L of TSB, 6 g/L of yeast extract, 1 g/L of esculin, 10 g/L of LiCl, 2 g/L of sodium pyruvate, 0.1 g/L of ferric ammonium citrate, 8 g/L of MOPS free acid, 14.2 g/L of MOPS, sodium, 5 g/L of beef extract, and 1% of a vitamin mix containing about 0.1 mg/L of riboflavin, about 1.0 mg/L of thiamine and about 1.0 mg/L of biotin, 10 mg/L of polymyxin B, and 20 mg/L of ceftazidime, and 5 mg/L acrifalvine, at 35° C. for 22 hours. Each MPN (most probable number) tube was cultivated in an enriched UVM-1 medium (5 g/L of proteose peptone, 5 g/L of tryptone/casein dig., 5 g/l of beef extract, 5 g/L of yeast extract, 20 g/L of NaCl, 12 g/L of Na₂HPO₄ 2H₂O, 1.35 g/L of KH₂PO₄, 1 g/L of esculin, 0.012 g/L of acroflavine, and 0.02 g/L of nalidixic acid) at 30° C. for 24 hours. PCR was carried out under the same conditions as in Tables 1 and 2. The PCR results are shown in Tables 5.

Whole milk was inoculated with L. ivanovii to concentrations of about 1 cfu/25 ml and about 5 cfu/25 ml. After incubation of the samples at 4° C. for 24 hours, each sample was cultivated in an enriched medium A2.2 medium (propriety formulation containing 30 g/L of TSB, 6 g/L of yeast extract, 1 g/L of esculin, 10 g/L of LiCl, 2 g/L of sodium pyruvate, 0.1 g/L of ferric ammonium citrate, 8 g/L of MOPS free acid, 14.2 g/L of MOPS, sodium, 5 g/L of beef extract, and 1% of a vitamin mix containing about 0.1 mg/L of riboflavin, about 1.0 mg/L of thiamine and about 1.0 mg/L of biotin, 10 mg/L of polymyxin B, and 20 mg/L of ceftazidime, and 5 mg/L acrifalvine, at 35° C. for 22 hours. Each MPN (most probable number) tube was cultivated in an enriched UVM-1 medium (5 g/L of proteose peptone, 5 g/L of tryptone/casein dig., 5 g/l of beef extract, 5 g/L of yeast extract, 20 g/L of NaCl, 12 g/L of Na₂HPO₄ 2H₂O, 1.35 g/L of KH₂PO₄, 1 g/L of esculin, 0.012 g/L of acroflavine, and 0.02 g/L of nalidixic acid) at 30° C. for 24 hours. PCR was carried out under the same conditions as in Tables 1 and 2. Each MPN tube was cultivated in an enriched UVM-1 medium at 30° C. for 24 hours. The PCR results are shown in Table 5.

TABLE 5
Detection of Listeria in contaminated liquid egg and milk

| | Detection (Cp values) | | | |
|---|---|---|---|---|
| | Liquid Egg | | Milk with | |
| Sample | with L. innocua | | L. ivanovii | |
| 1 | 31.01 | 31.21 | N/D | 36.99 |
| 2 | 33.76 | 33.33 | N/D | 37.07 |
| 3 | N/D | 32.32 | 36.71 | N/D |
| 4 | 31.88 | 40.28 | 31.71 | 36.59 |
| 5 | N/D | 34.42 | 41.07 | N/D |
| 6 | N/D | 31.14 | N/D | 39.59 |
| 7 | 31.11 | 31.34 | N/D | N/D |
| 8 | 41.84 | N/D | N/D | N/D |
| 9 | 27.57 | 28.5 | N/D | N/D |
| 10 | 32.46 | N/D | N/D | N/D |
| MPN | <1 cfu/ | 1.525 cfu/25 mL | 2.75 cfu/ | 1.85 cfu/25 mL |
| | 25 mL | | 25 mL | |

N/D: Not detected.

These results show that the real-time PCR assay in the presence of a primer pair of SEQ ID NOs. 3 and 7 and a probe of SEQ ID NO. 12 was able to detect trace levels (typically close to 1 cfu/25 g or ml of food) of Listeria species in the contaminated liquid egg and milk.

(2) Specific Detection of Listeria spp. in Contaminated Deli Turkey Meat, and on Stainless Steel Surface and Polypropylene Surface

Deli Turkey (ham) was inoculated with L. seeligeri to concentrations of about 2 cfu/25 g and about 4 cfu/25 g. After incubation of the samples at 4° C. for 24 hours, each sample was cultivated in an enriched medium A2.2 medium at 35° C. for 24 hours. Each MPN tube was cultivated in an enriched UVM-1 medium at 30° C. for 24 hours.

Contaminated stainless steel surface samples were prepared as follows. L. welshimeri was diluted with 0.5% non-fat milk to concentrations of 2 cfu and 10 cfu. 1 mL of each dilution was inoculated on a 4 inch×4 inch stainless steel surface and air-dried overnight at room temperature. The contaminant on each sample surface was collected with a phosphate buffered saline (PBS)-soaked sponge and then cultivated in an enriched medium A2.2 at 35° C. for 24 hours.

L. grayi was diluted with 0.5% non-fat milk to concentrations of 1 cfu and 10 cfu. 1 mL of each dilution was inoculated on a 4 inch×4 inch polyethylene film surface and air-dried overnight at room temperature. The contaminant on each sample surface was collected with a phosphate buffered saline (PBS)-soaked sponge and then cultivated in an enriched medium A2.2 at 35° C. for 24 hours.

PCR was carried out under the same conditions as in Tables 1 and 2. The PCR results are shown in Table 6.

TABLE 6
Detection of Listeria in contaminated ham and on environmental surfaces.

| | Detection of Listeria (Cp values) | | | | | |
|---|---|---|---|---|---|---|
| | HAM with | | Stainless steel with | | Polyproplyene | |
| Sample | L. seeligeri | | L. welshimeri | | with L. grayi | |
| 1 | N/D | N/D | N/D | 37.17 | N/D | N/D |
| 2 | N/D | 39.75 | N/D | 32.29 | N/D | 39 |
| 3 | N/D | N/D | 39.48 | 34.79 | N/D | 38.64 |
| 4 | N/D | N/D | N/D | N/D | 38.68 | 36.11 |
| 5 | N/D | 41.59 | 39.02 | N/D | N/D | N/D |
| 6 | 39.5 | N/D | N/D | 38.66 | 40.87 | N/D |
| 7 | N/D | N/D | N/D | 36.2 | N/D | 37.90 |
| 8 | N/D | N/D | N/D | 40.83 | N/D | 37.27 |
| 9 | N/D | N/D | N/D | N/D | 38.56 | 36.45 |
| 10 | 38.08 | N/D | N/D | N/D | 39.4 | 39.45 |
| MPN | 0.75 cfu/ | <1 cfu/ | 0.2 cfu/ | 0.83 cfu/4 | 1.30 cfu/ | 1.43 |
| | 25 g | 25 g | 4 inch² | inch² | 4 inch² | cfu/ |
| | | | | | | 4 inch² |

N/D: Not detected.

These results show that the real-time PCR assay in the presence of a primer pair of SEQ ID NOs. 3 and 7 and a probe of SEQ ID NO. 12 was able to detect the presence of Listeria species in the contaminated ham, and on contaminated environmental surfaces like stainless steel and polypropylene at a high sensitivity.

(3) Specific Detection of Listeria spp. on Contaminated Ceramic Tile, Rubber and Ground Beef

L. monocytogene was diluted with 0.5% non-fat milk to concentrations of 1 cfu and 10 cfu/mL. 1 mL of each dilution was inoculated on a 10×1 inch² ceramic tile surface and air-dried overnight at room temperature. The contaminant on each sample surface was collected with a phosphate buffered saline (PBS)-soaked sponge and then cultivated in 10 mL of an enriched medium A2.2 or UVM-1 medium at 35° C. or 30° C. for 24 hours.

L. innocua was diluted with 0.5% non-fat milk to concentrations of 3.3 and 33 cfu/mL. 1 mL of each dilution was inoculated on a 10×1 inch² rubber surface and air-dried overnight at room temperature. The contaminant on each sample surface was collected with a phosphate buffered saline (PBS)-soaked sponge and then cultivated in 10 mL of enriched medium A2.2 or UVM-1 medium at 35° C. or 30° C. for 24 hours.

PCR was carried out under the same conditions as in Tables 1 and 2. The PCR results are shown in Table 7.

TABLE 7
Detection of Listeria on contaminated environmental surfaces.

| | Detection of Listeria (Cp values) | | | | | | | |
|---|---|---|---|---|---|---|---|---|
| | Ceramic Tile with L. monocytogene | | | | Rubber with L. innocua | | | |
| | A2.2 | | UVM-1 | | A2.2 | | UVM-1 | |
| Sample | 1 cfu | 10 cfu | 1 cfu | 10 cfu | 3.3 cfu | 33 cfu | 3.3 cfu | 33 cfu |
| 1 | 17.62 | 18.68 | N/D | 31.03 | N/D | N/D | N/D | N/D |
| 2 | 17.16 | 20.09 | 35.22 | 35.94 | 40.94 | N/D | N/D | N/D |
| 3 | 19.83 | 17.06 | 38.94 | 31.08 | 40.46 | N/D | N/D | N/D |
| 4 | 17.85 | 17.28 | N/D | 36.97 | N/D | 40.47 | N/D | N/D |
| 5 | 16.97 | 16.91 | 35.5 | 35.37 | 40.81 | 39.79 | N/D | 40.51 |
| 6 | N/D | 17.38 | 32.73 | 34.51 | N/D | N/D | N/D | N/D |
| 7 | N/D | 19.63 | N/D | 39.87 | N/D | N/D | N/D | N/D |
| 8 | 17.78 | 21.27 | 38.12 | 40.84 | N/D | N/D | N/D | N/D |
| MPN | 0.1 cfu/ | 1 cfu/ | 0.1 cfu/ | 1 cfu/ | 0.33 cfu/ | 3.3 cfu/ | 0.33 cfu/ | 3.3 cfu/ |
| | inch² | inch² | inch² | inch² | inch² | inch² | inch² | inch² |

Referring to Table 7, it is confirmed that the real-time PCR assay in the presence of the primer set and a probe according to an embodiment of the invention detects the presence of Listeria species in the contaminated ceramic tile and rubber at a high sensitivity. Also, the results show that the A2.2 medium is better than the UVM-1 medium in terms of Listeria growth enhancing efficiency.

Ground beef was inoculated with L. monocytogenes to concentrations of about 2 cfu/25 g (Set A) and about 4 cfu/25 g (Set B), and then incubated at 4° C. for 30 hours. Then, each sample was cultivated in an enriched medium A2.2 medium at 35° C. for 24 hours. Each MPN tube was cultivated in an enriched UVM-1 medium at 30° C. for 24 hours. PCR was carried out under the same conditions as in Tables 1 and 2. The PCR results are shown in Table 8.

TABLE 8
Detection of Listeria in contaminated ground beef.

Detection of
Listeria (Cp values)

| Sample ID | Set A | Set B |
|---|---|---|
| 1 | 33.46 | 34.46 |
| 2 | 32.19 | 35.96 |
| 3 | N/D | 35.81 |
| 4 | 39.19 | N/D |
| 5 | 36.3 | 35.85 |
| 6 | N/D | 33.54 |
| 7 | N/D | N/D |
| 8 | N/D | 29.75 |
| 9 | N/D | N/D |
| 10 | 33.68 | 34.4 |
| MPN | <1 cfu/25 g | 0.75 cfu/25 g |

N/D: Not detected.

Referring to Table 8, it is confirmed that the real-time PCR assay in the presence of the primer set and a probe according to an embodiment of the invention detects the presence of Listeria species in the contaminated ground beef at a high sensitivity. Also, uncontaminated ground beef was found negative (not shown).

(4) Specific Detection of Listeria spp. in Contaminated Smoked Ham

Smoked ham was inoculated with L. monocytogenes to a concentration of about 3 cfu/25 g, and then incubated at 4° C. for 48 hours. Then, the sample was cultivated in an enriched medium A2.2 medium at 35° C. for 24 hours. Each MPN tube (0.1 g, 1 g, and 10 g) was cultivated in an enriched UVM-1 medium at 30° C. for 24 hours. Each sample was diluted in dilution ratios of 1:5 (20 μl:80 μl) and 1:10 (10μl:90 μl) between the contaminated sample and the non-contaminated sample. PCR was carried out under the same conditions as in Tables 1 and 2. The PCR results are shown in Table 9.

TABLE 9
Detection of Listeria in contaminated ham.

| Sample | Undiluted | 1:5 dilution | 1:10 dilution |
|---|---|---|---|
| 1 | 30.08 | 30.95 | 31.85 |
| 2 | 28.15 | 29.1 | 29.97 |
| 3 | 31.07 | 32.47 | 32.61 |
| 4 | 39.74 | 30.46 | 31.56 |
| 5 | N/D | N/D | N/D |
| 6 | N/D | N/D | N/D |
| 7 | N/D | N/D | N/D |
| 8 | 29.66 | 29.95 | 30.75 |
| MPN (/25 g) | 0.75 cfu/25 g | | |

N/D: Not detected.

Referring to Table 9, it is confirmed that the real-time PCR assay in the presence of the primer set and a probe according to an embodiment of the invention detects the presence of Listeria species in the contaminated ham at a high sensitivity. Ability of the assay to detect Listeria species in diluted samples proves it suitable for pooled samples.

(5) Specific Detection of Listeria spp. in Rubber Contaminated with Listeria and E. coli

L. monocytogenes was diluted with 0.5% non-fat milk to concentrations of 1 cfu/100 μL and 10 cfu/100 μL. These dilutions contained 8 cfu and 80 cfu of E. coli, respectively, per 100 μL. 1000 μL of each suspension was inoculated on a 10×1 inch² inch rubber surface and air-dried overnight at room temperature. The contaminant on each sample surface was collected with a DE-soaked sponge and then cultivated in 10 mL of an enriched medium A2.2 or UVM-1 medium at 35° C. or 30° C. for 24 hours. PCR was carried out under the same conditions as in Tables 1 and 2. The PCR results are shown in Table 10.

Composition of the DE broth used is as follows:
Pancreatic Digest of Casein . . . 5.0 g
Yeast Extract . . . 2.5 g
Dextrose . . . 10.0 g
Sodium Thioglycollate . . . 1.0 g
Sodium Thiosulfate . . . 6.0 g
Sodium Bisulfite . . . 2.5 g
Polysorbate 80 . . . 5.0 g
Lecithin . . . 7.0 g
Bromcresol Purple . . . 0.02 g

TABLE 10
Detection of Listeria on contaminated rubber surfaces.

| Results | |
|---|---|
| Detection of Listeria (Cp values) | |
| Enrichment medium | |
| A2.2 | UVM-I |

| | L. mono. | L. mono | L. mono. | L. mono. |
|---|---|---|---|---|
| | 1cfu + | 10 cfu + | 1cfu + | 10 cfu + |
| | E. coli | E. coli | E. coli | E. coli |
| Sample | 8 cfu | 80 cfu | 8 cfu | 80 cfu |
| 1 | 38.83 | 32.7 | 37.81 | 43.14 |
| 2 | 30.24 | 30.35 | 41.49 | 45 |
| 3 | 22.11 | N/D | 38.9 | 41.69 |
| 4 | 27.68 | N/D | 40.89 | 45 |
| 5 | 27.59 | 22.65 | 40.58 | N/D |
| 6 | N/D | 34.86 | 39.03 | N/D |
| 7 | N/D | 29.87 | 39.91 | N/D |
| 8 | 22.44 | 42.45 | N/D | N/D |
| 9 | N/D | 25.72 | N/D | 43.38 |
| 10 | N/D | 27.13 | 41.68 | N/D |
| MPN | 1 cfu/in² | 10 cfu/in² | 1 cfu/in² | 10 cfu/in² |

N/D: Not detected.

Referring to Table 10, it is confirmed that the real-time PCR assay in the presence of the primer set and a probe according to an embodiment of the invention detects at a high sensitivity the presence of Listeria species in rubber contaminated with L. monocytogenes and E. coli. The results in Table 10 also show that the A2.2 medium is better than the UVM-1 medium in terms of Listeria growth enhancing efficiency.

### EXAMPLE 3

### Specific Detection of Listeria spp. in Contaminated Samples by RT-PCR

(1) Ceramic Tile Surface Contaminated with L. monocytogene

L. monocytogene was diluted with 0.5% non-fat milk to a concentration of 16 cfu/100 μL. 80 μL of the suspension was inoculated on a 10×1 inch² ceramic tile surface and air-dried overnight at room temperature. The contaminant on the sample surface was collected with a PBS or DE-soaked sponge and then cultivated in 8 mL of a pre-warmed brain-heart infusion (BHI) medium at 35° C. for 6 hours. Then, 1 mL of the culture products was inoculated into 9 ml of a UVM-1 medium and further incubated at 30° C. for 18 hours. Separately, 1 ml of the culture products was inoculated onto 9 ml of BHI medium at 35° C. for 6 hours.

The culture products from the 6-hour cultivation in the BHI medium was used for reverse transcriptase (RT) reaction (700 μL of enriched culture products+100 μL of 1×ZAC (1% CHAPS, 2.5 mg/mL sodium azide, and 100 mM Tris (pH 8))+10 μL of proteinase K). A TZ lysis buffer (2.0% Triton X-100 and 2.5 mg sodium azide per 1 ml of 0.1M Tris-HCl buffer, pH 8.0) was used for other samples.

The reverse transcription reaction was induced as follows. 7.9 μL of DEPC-water, 0.1 μL of a 20 μM reverse primer, 1 μL of 10 mM dNTP and 1 μL of lysate were mixed. The used reverse primer was SEQ ID NO: 7. The mixture was incubated at 65° C. for 5 minutes, and then placed on ice for 2 minutes.

2 μL of a 10×RT buffer, 4 μL of a 25 mM MgCl₂, 24, of a 0.1 M DTT, 1 μL of RNase HII (40 U/ml) and 1 μL of Superscript III (1 U/μL, reverse transcriptase) were added to the mixture.

After incubation at 50° C. for 50 minutes, the mixture was further incubated at 85° C. for 5 minutes, and then cooled to 4° C. 2 μL of the RT products was mixed with a PCR mixture for PCR. The PCR conditions and the PCR mixture composition were the same as in Tables 1 and 2. Tables 11 and 12 represents the Cp values obtained from the RT-PCR and PCR, respectively.

TABLE 11
Detection of Listeria (collected with PBS-soaked sponges)

| | Results: | | |
|---|---|---|---|
| | Detection of Listeria (Cp values) | | |
| | RT-6 hour | A2.2 medium 24 hour | UVM medium 24 hour |
| Sample | (RT + PCR) | (PCR only) | (PCR only) |
| 1 | N/D | 23.03 | 39.9 |
| 2 | 37.29 | 20.48 | N/D |
| 3 | 32.84 | N/D | N/D |
| 4 | N/D | 29.2 | N/D |
| 5 | 28.28 | 25.92 | 39.65 |
| 6 | 37.57 | 22.84 | N/D |
| 7 | N/D | 23.22 | 37.67 |
| 8 | N/D | N/D | 39.76 |
| 9 | N/D | 30.46 | N/D |
| 10 | N/D | 25.03 | N/D |

| | |
|---|---|
| MPN | 0.13 cfu/inch² |

N/D: Not detected.

TABLE 12
Detection of Listeria (collected with DE-soaked sponges)

| | Detection of Listeria (Cp values) | | |
|---|---|---|---|
| | RT-6 hour | A2.2 medium 24 hour | UVM medium 24 hour |
| Sample | (RT + PCR) | (PCR only) | (PCR only) |
| 1 | 34.3 | 24.33 | N/D |
| 2 | N/D | N/D | N/D |
| 3 | 38.49 | 38.51 | N/D |
| 4 | N/D | 25.69 | 39.66 |
| 5 | N/D | 33.12 | N/D |
| 6 | N/D | 23.27 | 38.16 |
| 7 | 35.76 | N/D | N/D |
| 8 | 27.28 | 24.66 | N/D |
| 9 | N/D | 30.56 | N/D |
| 10 | N/D | 21.66 | N/D |

| | |
|---|---|
| MPN | 0.13 cfu/inch² |

As is apparent from Tables 11 and 12, Listeria spp. can be detected rapidly and sensitively by a shorter enrichment protocol RT-PCR, compared to the conventional 24-hour enrichment protocol.

(2) Rubber Surface Contaminated with L. monocytogene

L. monocytogene was diluted with 0.5% non-fat milk to a concentration of 2.25 cfu/100 μl, and E. coli was diluted in the same manner to a concentration of 23 cfu/100 μL. 100 μL of the suspension was inoculated on a 1 inch×1 inch rubber surface and air-dried overnight at room temperature. The contaminant on the sample surface was collected with a DE-soaked cotton swab, and then cultivated in 8 ml of a preheated enriched BHI medium at 35° C. for 6 hours, in 10 ml of a UVM-1 medium at 30° C. for 24 hours, or in 10 ml of A2.2 medium at 35° C. for 24 hours.

The culture products from the 6-hour cultivation in the BHI medium were used for reverse transcriptase (RT) reaction (700 μL of enriched culture products+100 μL of 1×ZAC+10 μL of proteinase K). A TZ lysis buffer (2.0% Triton X-100 and 2.5 mg sodium azide per 1 ml of 0.1M Tris-HCl buffer, pH 8.0) was used for other samples.

For RT reaction of 20 μL of the sample, 7.9 μL of DEPC-water, 0.1 μL of a 20 μM reverse primer, 1 μL of 10 mM dNTP and 1 μL of lysate were mixed. The used forward and reverse primers were SEQ ID NO:3 and SEQ ID NO:7, respectively. The mixture was incubated at 65° C. for 5 minutes, and then placed on ice for 2 minutes. 2 μL of a 10×RT buffer, 4 μL of a 25 mM MgCl₂, 2 μL, of a 0.1 M DTT, 1 μL of RNase HII (40 U/ml) and 1 μL of Superscript III (200 U/μL, reverse transcriptase) were added to the mixture. After incubation at 50° C. for 50 minutes, the mixture was further incubated at 85° C. for 5 minutes, and then cooled to 4° C. 2 μL of the RT products was mixed with a PCR mixture for PCR. The PCR conditions and the PCR mixture composition were the same as in Tables 1 and 2. Table 13 represents the Cp values obtained from the RT-PCR.

TABLE 13
Detection of Listeria (collected with DE-soaked sponges)

| | Results: | | |
|---|---|---|---|
| | Detection of Listeria (Cp values) | | |
| | | | UVM medium 24 |
| | RT-6 hour | A2.2 medium 24 hour | hour |
| Sample | (RT + PCR) | (PCR only) | (PCR only) |
| 1 | N/D | 39.06 | 37.99 |
| 2 | 38.13 | N/D | N/D |
| 3 | 39.2 | 39.05 | 38.8 |
| 4 | 41.5 | 39.37 | 39.44 |
| 5 | 42.3 | 38.99 | 38.62 |
| 6 | 38.1 | N/D | N/D |
| 7 | N/D | N/D | N/D |
| 8 | N/D | 38.99 | 37.54 |
| 9 | N/D | 39.06 | 37.94 |
| 10 | N/D | 39.07 | 38.73 |
| 11 | N/D | 38.18 | N/D |
| 12 | 37.5 | N/D | 36.92 |
| 13 | N/D | 39.03 | 38.5 |
| 14 | N/D | 38.91 | 39.08 |
| 15 | N/D | 39.26 | N/D |

| | |
|---|---|
| MPN | 2.25 cfu/inch² |

N/D: Not detected.

As is apparent from Table 13, Listeria spp. can be detected rapidly by RT-PCR with high sensitivity.

### EXAMPLE 4

### Specific Detection of Listeria spp. in Contaminated Sample by One-Step RT-PCR

(1) A synthetic target 23S RNA of Listeria spp. was serially diluted by 10 folds from 2×10⁷ copies/μL to 20 copies/μL. One-step RT-PCR was performed using the RNA molecules as a template. The composition of 25 μL of the reaction mixture and the RT-PCR conditions were the same as in Tables 14 and 15, respectively.

TABLE 14
RT-PCR mixture composition (in each well μL)

| Reaction I | | Reaction II | |
|---|---|---|---|
| | (μL per | | (μL per |
| | 25 μL | | 25 μL |
| Component | reaction) | Component | reaction) |
| 10x Buffer 6 | 2.5 | 10x ICAN | 2.5 |
| Forward F (20 uM) | 0.5 | Forward F (20 uM) | 0.5 |
| Reverse R (20 uM) | 0.5 | Reverse R (20 uM) | 0.5 |
| CC probe (5 uM) | 1 | CC probe (5 uM) | 1 |
| dNTP (25 mM) | 0.4 | dNTP (25 mM) | 0.4 |
| RT-Taq Enzyme Mix | 1 | RT-Taq Enzyme Mix | 1 |
| UNG (10 unit/μL) | 0.1 | UNG (10 unit/μL) | 0.1 |
| HotStart Pfu | 0.5 | HotStart Pfu RNaseHII | 0.5 |
| RNaseHII (5 unit/μL) | | (5 unit/μL) | |
| Template RNA | 0.5 | Template RNA | 0.5 |
| Water | 18.00 | Water | 18.00 |

In Table 14, Buffer 6 contains 4 mM magnesium acetate, 50 mM potassium acetate, 50 mM Tris-acetate (pH 8.6), 1 mM DTT. The forward and reverse primers and the CC probe were oligonucleotides of SEQ ID NOs. 3, 7 and 12, respectively. HotStart Pfu RNase HII was used which is a reversibly modified and thermostable RNase HII enzyme that starts to denaturate at RT temperature and becomes active at high temperatures. The modification was achieved by reversible formaldehyde crosslinking. Two buffers were used for the crosslinking: a crosslinking buffer containing 20 mM HEPES, 200 mM KC at pH 7.9, and 1 mM EDTA; and a 2×RNase HII storage buffer containing 100 mM Tris-HCl (pH 8.0), 200 mM NaCl, and 0.2 mM EDTA

For purpose of preparing HotStart Pfu RNase HII, 2 μL of a Pfu RNase HII (25 mg/ml, about 50 OD) was diluted with 47 μL of the crosslinking buffer (1.25 mg/ml, about 2.5 OD). 10 mL of the diluted Pfu RNase HII (1.25 mg/ml, about 2.5 OD on ice), 7.25 ml of water, and 0.75 ml of a 13.8% formaldehyde (in water) were mixed to prepare 18 mL of a final reaction mixture (Final formaldehyde concentration was 0.58%). Then, the reaction mixture was incubated at 37° C. for 30 minutes. The reaction mixture was placed on iced, and 2 μL of 2M Tris-HCl (pH 8.0) was added to the reaction mixture. After completion of the reaction, the reaction mixture was purified using a G50 microspin column pre-equilibrated with the 2×RNase HII storage buffer, and was then diluted with an equal amount of glycerol and stored at −20° C.

The modified RNase HII lost its activity at 50° C. but was reactivated when heated to 95° C.

TABLE 15
PCR reaction conditions

| Step | Temp (° C.) | Time (min) | Cycles |
|---|---|---|---|
| RT | 50 | 30 | 1 |
| Denaturation | 95 | 15 | 1 |
| Cycles | 94 | 0.25 | 50 |
| | 60 | 0.67 | |

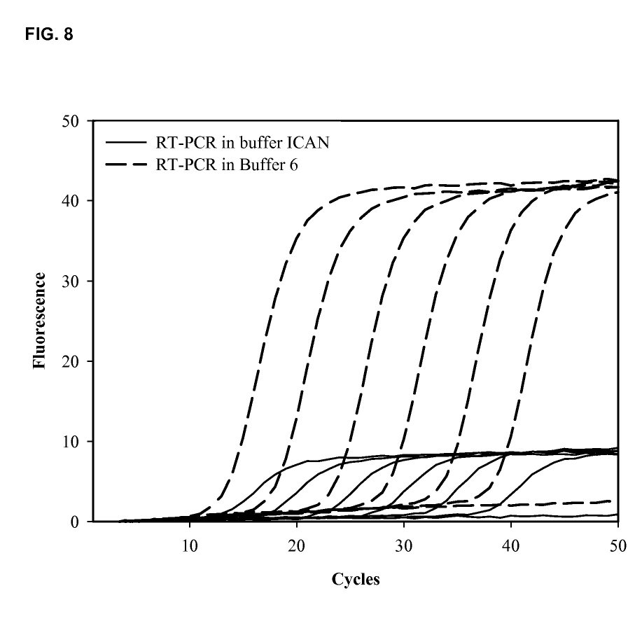

Results are shown in FIG. 8. As a result of the RT-PCR, Listeria was detected down to 10 copies per reaction under the conditions of both Reaction I and Reaction II. However, the fluorescence intensity in Reaction I was much higher than that in Reaction II, indicating a higher probe cleavage kinetics in Reaction I.

### (2) Different RT-PCR Buffers

By following the same procedure, except employing the buffers shown in Table 16 below for the RT-PCR, RT-PCR were performed.

TABLE 16
RT-PCR recipe in each well (μL)

| | |
|---|---|
| Reaction I | Reaction II |
| (AgPath One-Step | (Platinum Tfi |
| RT-PCR) | One-Step RT-PCR) |
| μL/25 μL reaction | μL/25 μL reaction |

| | | | |
|---|---|---|---|
| 2x AgPath Buffer | 12.5 | 2x Tfi Buffer | 12.5 |
| Forward F (20 μM) | 0.5 | Forward F (20 μM) | 0.5 |
| Reverse R (20 μM) | 0.5 | Reverse R (20 μM) | 0.5 |
| CC probe (5 μM) | 1 | CC probe (5 μM) | 1 |
| RT-Taq Enzyme Mix | 1 | RT-Taq Mix Enzyme | 0.625 |
| HotStart Pfu RNaseHII | 0.5 | HotStart Pfu RNaseHII | 0.5 |
| (5 unit/μL) | | (5 unit/μL) | |
| Template DNA | 2 | Template DNA | 2 |
| Water | 7.00 | Water | 7.35 |

AgPath One-Step RT-PCR kit and Tfi One-Step RT-PCR, which contain proprietary formulation, were obtained from Life Tech.

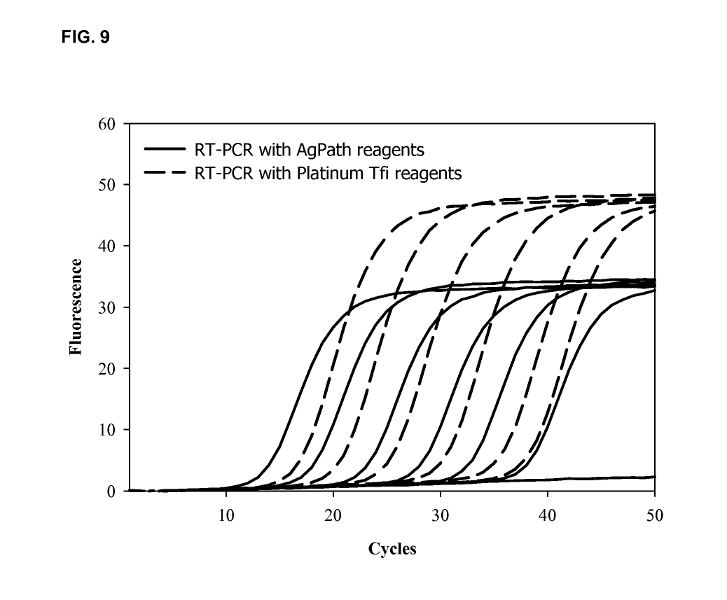

FIG. 9 illustrates the results of RT-PCR conducted using the Tfi buffer and the AgPath buffer, respectively. The results of FIG. 9 indicate that one step RT-PCR using the primer pair of SEQ ID NOs. 3 and 7 and the probe of SEQ ID NO. 12 is suitable to efficiently detect Listeria spp. in a sample with a sensitivity of 10 copies per reaction.

### EXAMPLE 5

### Increase in Sensitivity by Enrichment Culture

Overnight grown L. monocytogenes was diluted in 10-fold with PBS to a concentration of about 1 cfu/100 μL. Then, 100 μL or 1 mL of the diluted L. mono was added to 15 mL fresh BHI broth, and incubated at 35° C. for 6 hours without shaking. Four replicates were tested for each dilution levels.

After 6 hours, 700 μL enrichment was lysed and 1 μL lysate was used as template in Invitrogen SUPERSCRIPT III™ reaction according to the manufacturer's protocol. 2 μL of cDNA was tested in PCR/CataCleave reactions. The PCR conditions and the PCR mixture composition were the same as in Tables 1 and 2.

In the meantime, 100 μL enrichment was plated, and cell counts next day was 10 cfu/mL.

It was shown that the assay was able to detect a cell concentration of 10 cfu/mL at a Cp of 39.47±0.92. Also, it was observed that a 1:10 dilution of the lysate before reverse transcriptase (RT) reaction helped increase sensitivity of the test.

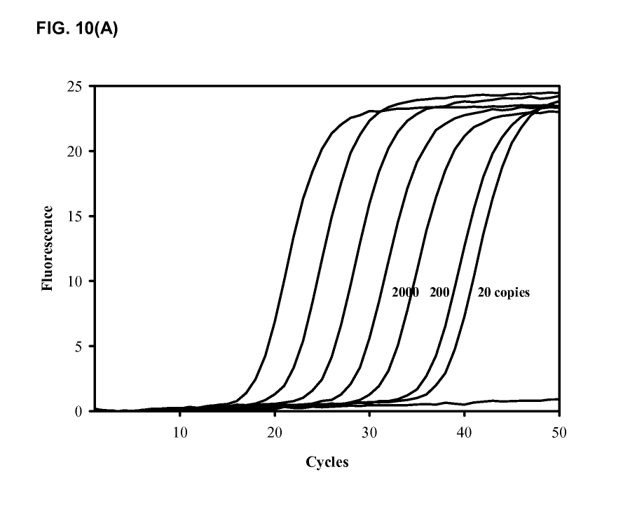

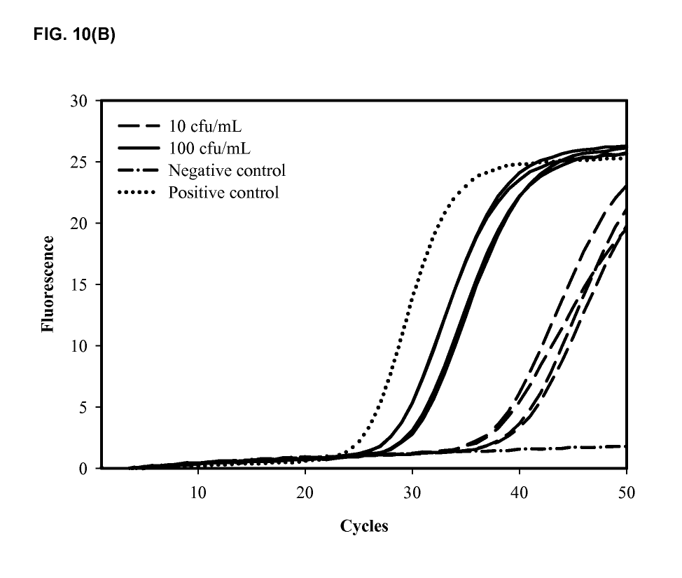

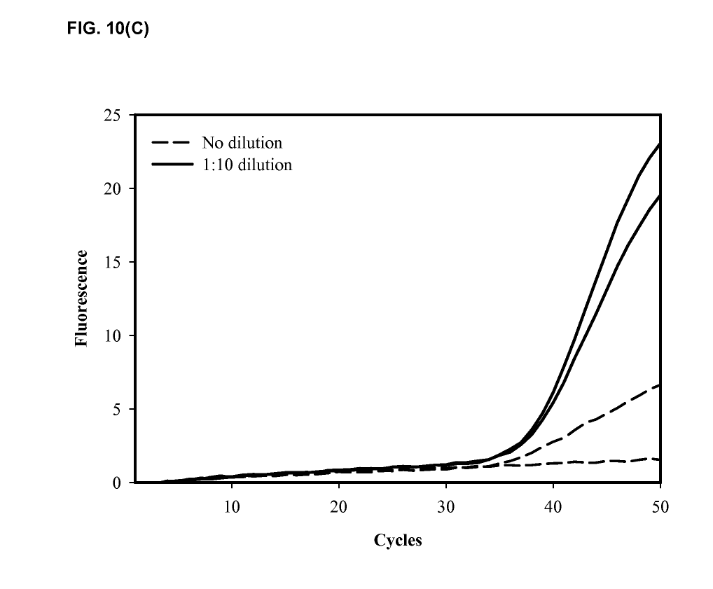

Results are shown in FIG. 10(A)-10(C). FIG. 10(A) shows the amplification curve of isolated target RNA molecules, which shows as low as 20 copies of target RNA molecules could be detected when the sample was enriched before RT PCR. FIG. 10(B) shows the amplification curve of enriched cell suspension of the sample. The enrichment culture increased about 300-500 times of sensitivity of detection of target RNA molecule in cell suspension. Also, when the enriched culture is diluted with water before conducting RT PCR, the enrichment showed minimal inhibition of RT PCR (FIG. 10(C)).

Accordingly, the enrichment culture for about 6 hours before RNA extraction enables a surprisingly rapid detection of Listeria sp. In an embodiment, a total of about 8 hour from the collection of a sample to finish the test.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

What is claimed is:

1. A composition comprising:
a first oligonucleotide of the sequence of SEQ ID NO: 19:
X₁CCAAGCAGTGAGTGTGAGAAX₂ (SEQ ID NO:19), wherein X₁ at position 1 is absence or T, and X₂ at position 22 is absence or G, and
a second oligonucleotide of the sequence of SEQ ID NO: 20:
X₁X₁GACAGCGTGAAATCAGGX₃X₃X₄ (SEQ ID NO: 20), wherein X₁s at positions 1 and 2 are each absence or T; X₃ at position 20 and 21 are absence or A; and X₄ at position 22 is absence or C.

2. The composition according to claim 1, wherein the number of nucleotide residues in the first oligonucleotide of SEQ ID NO: 19 is 20 or 21.

3. The composition according to claim 2, wherein the number of nucleotide residues in the second oligonucleotide of SEQ ID NO: 20 is 18-21.

4. The composition according to claim 1, wherein the first oligonucleotide is one or more selected from the group of oligonucleotides of SEQ ID NOs: 1-3:

| | |
|---|---|
| CCAAGCAGTGAGTGTGAGAAG, | (SEQ ID NO: 1) |
| CCAAGCAGTGAGTGTGAGAA, | (SEQ ID NO: 2) |
| and | |
| TCCAAGCAGTGAGTGTGAGAA. | (SEQ ID NO: 3) |

5. The composition according to claim 1, wherein the second oligonucleotide is one or more selected from the group of oligonucleotides of SEQ ID NOs: 5-9:

| | |
|---|---|
| TGACAGCGTGAAATCAGGAAC, | (SEQ ID NO: 5) |
| TTGACAGCGTGAAATCAGG, | (SEQ ID NO: 6) |
| TGACAGCGTGAAATCAGGA, | (SEQ ID NO: 7) |
| TGACAGCGTGAAATCAGGA, | (SEQ ID NO: 8) |
| and | |
| GACAGCGTGAAATCAGGA. | (SEQ ID NO: 9) |

6. The composition according to claim 1, further comprising a third oligonucleotide comprising a DNA sequence and an RNA sequence, said third oligonucleotide being the sequence of SEQ ID NO: 21 or SEQ ID NO: 22:
TGCGAAGACTGAGCTGTGATGG (SEQ ID NO: 21), wherein at least one of nucleotides at positions 8 and 9 are a ribonucleotide, and
CCATCACAGCrUrCrArUGCTTCGC (SEQ ID NO. 22), wherein at least one of “rU,” “rC,” “rA,” and “rU” at positions 11, 12, 13, and 14, respectively, is a ribonucleotide.

7. The composition according to claim 6, wherein the third oligonucleotide is one or more selected from the group consisting of oligonucleotides of SEQ ID NOs: 10-14:
TGCGAAGCrATGAGCTGTGATGG (SEQ ID NO: 10), wherein “rA” at position 9 is a ribonucleotide,
TGCGAAGrCATGAGCTGTGATGG (SEQ ID NO: 11), wherein “rC” at position 8 is a ribonucleotide,
CCATCACAGCTCArUGCTTCGC (SEQ ID NO: 12), wherein “rU” at position 14 is a ribonucleotide,
CCATCACAGCTrCrArUGCTTCGC (SEQ ID NO: 13), wherein “rC,” “rA,” and “rU” at positions 12, 13, and 14, respectively, are a ribonucleotide; and
CCATCACAGCrUrCrArUGCTTCGC (SEQ ID NO: 14), wherein “rU,” “rC,” “rA,” and “rU” at positions 11, 12, 13, and 14, respectively, are a ribonucleotide.

8. The composition according to claim 6, wherein the third oligonucleotide is labeled with a detectable marker.

9. The composition according to claim 8, wherein the third oligonucleotide is labeled with a fluorescence resonance energy transfer (FRET) pair.

10. The composition according to claim 6, comprising the first oligonucleotide of SEQ ID NO. 3, the second oligonucleotide of SEQ ID NO. 7, and the third oligonucleotide of SEQ ID NO. 12.

11. A kit for detecting Listeria spp. in a sample, the kit comprising
(a) a first primer of the sequence of SEQ ID NO: 19:
X₁CCAAGCAGTGAGTGTGAGAAX₂ (SEQ ID NO:19), wherein X₁ at position 1 is absence or T, and X₂ at position 22 is absence or G;
(b) a second primer of the sequence of SEQ ID NO: 20:
X₁X₁GACAGCGTGAAATCAGGX₃X₃X₄ (SEQ ID NO: 20), wherein X₁s at positions 1 and 2 are each absence or T; X₃ at position 20 and 21 are absence or A; and X₄ at position 22 is absence or C; and
(c) a probe comprising an RNA sequence and a DNA sequence that are substantially complimentary to a target Listeria spp. gene, and coupled to a detectable label.

12. The kit according to claim 11, further comprising
(d) an amplifying activity for a PCR amplification of the target DNA sequence to produce a Listeria spp. PCR fragment; and
(e) an RNase H activity.

13. The kit according to claim 12, further comprising positive, internal, and negative controls.

14. The kit according to claim 13, further comprising uracil-N-glycosylase.

15. The kit according to claim 11, wherein the probe is coupled to a detectable label at both of its 3′- end and 5′-end.

16. The kit according to claim 15, wherein the detectable label is a fluorescent label.

17. The kit according to claim 16, wherein the probe is labeled with a FRET pair.

18. The kit according to claim 12, wherein the probe is linked to a solid support.

19. The kit according to claim 12, which further comprises an amplification buffer.

20. The kit according to claim 12, which further comprises an amplifying polymerase activity.

21. The kit according to claim 12, wherein the RNase H activity is the activity of a thermostable RNase H.

22. The kit according to claim 12, wherein the RNase H activity is a hot start RNase H activity.

23. The kit according to claim 11, wherein the first primer is one or more selected from the group of oligonucleotides of SEQ ID NOs: 1-3:

| | |
|---|---|
| CCAAGCAGTGAGTGTGAGAAG, | (SEQ ID NO: 1) |
| CCAAGCAGTGAGTGTGAGAA, | (SEQ ID NO: 2) |
| and | |
| TCCAAGCAGTGAGTGTGAGAA. | (SEQ ID NO: 3) |

24. The kit according to claim 11, wherein the second primer is one or more selected from the group of oligonucleotides of SEQ ID NOs: 5-9:

| | |
|---|---|
| TGACAGCGTGAAATCAGGAAC, | (SEQ ID NO: 5) |
| TTGACAGCGTGAAATCAGG, | (SEQ ID NO: 6) |
| TGACAGCGTGAAATCAGGA, | (SEQ ID NO: 7) |
| TGACAGCGTGAAATCAGGA, | (SEQ ID NO: 8) |
| and | |
| GACAGCGTGAAATCAGGA. | (SEQ ID NO: 9) |

25. The kit according to claim 11, wherein the probe comprises the sequence of SEQ ID NO: 21 or SEQ ID NO: 22:
TGCGAAGACTGAGCTGTGATGG (SEQ ID NO: 21), wherein at least one of nucleotides at positions 8 and 9 are a ribonucleotide, and
CCATCACAGCrUrCrArUGCTTCGC (SEQ ID NO. 22), wherein at least one of “rU,” “rC,” “rA,” and “rU” at positions 11, 12, 13, and 14, respectively, is a ribonucleotide.

26. The kit according to claim 11, wherein the probe is one or more selected from the group consisting of oligonucleotides of SEQ ID NOs: 10-14:
TGCGAAGCrATGAGCTGTGATGG (SEQ ID NO: 10), wherein “rA” at position 9 is a ribonucleotide,
TGCGAAGrCATGAGCTGTGATGG (SEQ ID NO: 11), wherein “rC” at position 8 is a ribonucleotide,
CCATCACAGCTCArUGCTTCGC (SEQ ID NO: 12), wherein “rU” at position 14 is a ribonucleotide,
CCATCACAGCTrCrArUGCTTCGC (SEQ ID NO: 13), wherein “rC,” “rA,” and “rU” at positions 12, 13, and 14, respectively, are a ribonucleotide; and
CCATCACAGCrUrCrArUGCTTCGC (SEQ ID NO: 14), wherein “rU,” “rC,” “rA,” and “rU” at positions 11, 12, 13, and 14, respectively, are a ribonucleotide.

27. A method of detecting Listeria spp. in a sample, the method comprising:
(a) amplifying a target nucleic acid of Listeria spp. in the sample to produce an increased number of copies of the target nucleic acid, the amplifying including hybridizing a first primer of SEQ ID NO: 19 and a second primer of SEQ ID NO: 20 to the target nucleic acid in the sample to obtain a hybridized product of the target nucleic acid and the primers, and extending the first and the second primers of the hybridized product using a template-dependent nucleic acid polymerase to produce an extended primer product;
(b) hybridizing the target nucleic acid to at least one probe oligonucleotide which is capable of being hybridized to the target nucleic acid to obtain a hybridized product of the target nucleic acid:probe oligonucleotide, wherein the probe comprises a DNA sequence and an RNA sequence and is coupled to a label;
(c) contacting the hybridized product of the target nucleic acid:the probe oligonucleotide to an RNase H to cleave the probes; and
(d) detecting an increase in the emission of a signal from the label on the probe, wherein the increase in signal indicates the presence of the Listeria spp. target nucleic acid in the sample.

28. The method according to claim 27, wherein the probe oligonucleotide is the oligonucleotide of SEQ ID NO: 21 or 22.

29. The method according to claim 28, wherein the probe oligonucleotide is selected from the group consisting of oligonucleotides of SEQ ID NOs: 10-14.

30. The method according to claim 27, wherein the label of the probe is a detectable marker.

31. The method according to claim 30, wherein the detectable marker is a fluorescence resonance energy transfer pair.

32. The method according to claim 27, wherein the amplifying is conducted using a method selected from the group consisting of Polymerase Chain Reaction, Ligase Chain Reaction, Self-Sustained Sequence Replication, Strand Displacement Amplification, Transcriptional Amplification System, Q-Beta Replicase, Nucleic Acid Sequence Based Amplification, Cleavage Fragment Length Polymorphism, Isothermal and Chimeric Primer-initiated Amplification of Nucleic Acid, and Ramification-extension Amplification Method.

33. The method according to claim 27, wherein the amplifying, the hybridizing and the contacting are simultaneously or sequentially carried out.

34. The method according to claim 27, further comprising cultivating the sample containing Listeria spp. in an enrichment medium before the amplifying, to enhance growth of the Listeria spp.

35. The method according to claim 34, wherein the enriched medium containing, per 1 L of distilled water, about 10 to about 40 g of tryptic soy broth, about 1 to about 10 g of yeast extract, and about 1 to about 10 g of lithium chloride.

36. The method according to claim 35, wherein the enriched medium further comprises at least one component selected from the group consisting of about 1 to about 10 g of beef extract, and/or a vitamin mix containing about 0.01 to about 0.5 mg of riboflavin, about 0.5 to about 1.5 mg of thiamine and about 0.01 to about 1.5 mg of biotin; about 1 to about 5 g of pyruvate or a salt thereof; and about 0.01 to about 1 g of ferric ammonium citrate.

37. The method according to claim 36, wherein the enrichment medium further comprises a buffer compound.

38. The method according to claim 37, wherein the buffer compound comprises 3-(N-morpholino)propanesulfonic acid (MOPS) and a sodium salt thereof.

39. The method according to claim 38, wherein the buffer compound comprises about 4 g of MOPS and about 7.1 g of sodium MOPS.

40. The method according to claim 34, wherein the enrichment medium comprises about 1 to about 10 mg of acriflavine, about 5 to about 15 mg of polymyxin B, and about 10 to about 30 mg of ceftazidime.

41. The method according to claim 34, wherein the enrichment medium comprises, per 1 L of distilled water, about 10 to about 40 g of tryptic soy broth, about 1 to about 10 g of yeast extract, about 1 to about 10 g of lithium chloride; about 1 to about 10 g of beef extract and/or a vitamin mix containing about 0.01 to about 0.5 mg of riboflavin, about 0.5 to about 1.5 mg of thiamine, and about 0.01 to about 1.5 mg of biotin; about 1 to about 5 g of pyruvate or a salt thereof; about 0.1 to about 1 g of ferric ammonium citrate; about 4 g of 3-(N-morpholino)propanesulfonic acid (MOPS) and about 7.1 g of sodium MOPS; and about 1 to about 10 mg of acriflavine, about 5 to about 15 mg of polymyxin B, and about 10 to about 30 mg of ceftazidime.

42. The method according to claim 34, wherein the enrichment medium does not comprise one of esculin or peptone, or both.

43. The method according to claim 34, wherein the enrichment medium comprises, per 1 L of distilled water, about 30 g of tryptic soy broth, about 6 g of yeast extract, about 1 to about 10 g of lithium chloride; about 5 g of beef extract and/or a vitamin mix containing about 0.1 mg of riboflavin, about 1.0 mg of thiamine, and about 1.0 mg of biotin; about 2 g of sodium pyruvate; about 0.2 g of ferric ammonium citrate; about 4 g of 3-(N-morpholino)propanesulfonic acid (MOPS) and about 7.1 g of sodium MOPS; and about 5 mg of acriflavine, about 10 mg of polymyxin B, and about 20 mg of ceftazidime.

44. The method according to claim 34, wherein the enrichment medium is a tryptic soy broth supplemented with a yeast extract, or a brain-heart infusion broth.

45. The method according to claim 27, wherein the sample is food or a surface wipe.

46. A method of detecting Listeria spp. in a sample, the method comprising:
(a) reverse transcribing the Listeria spp. target RNA in the presence of a reverse transcriptase activity and the reverse amplification primer to produce a target cDNA of the target RNA;
(b) amplifying the target cDNA sequence to produce an increased number of copies of the target nucleic acid, the amplifying including hybridizing a first primer of SEQ ID NO: 19 and a second primer of SEQ ID NO: 20 to the target cDNA to obtain a hybridized product of the target nucleic acid and the primers, and extending the first and the second primers of the hybridized product using a template-dependent nucleic acid polymerase to produce an extended primer product;
(c) hybridizing the target nucleic acid to at least one probe oligonucleotide which is substantially complimentary to the target cDNA to obtain a hybridized product of the target nucleic acid:probe oligonucleotide, wherein the probe comprises a DNA sequence and an RNA sequence and is coupled to a label;
(d) contacting the hybridized product of the target nucleic acid:probe oligonucleotide to an RNase H to cleave the probes; and
(e) detecting an increase in the emission of a signal from the label on the probe, wherein the increase in signal indicates the presence of the Listeria spp. target RNA in the sample.

47. The method according to claim 46, wherein the probe oligonucleotide is the oligonucleotide of SEQ ID NO: 21 or 22.

48. The method according to claim 47, wherein the probe oligonucleotide is selected from the group consisting of oligonucleotides of SEQ ID NOs: 10-14.

49. The method according to claim 46, wherein the label is a detectable marker.

50. The method according to claim 49, wherein the detectable marker is a fluorescence resonance energy transfer pair.

51. The method according to claim 46, wherein the amplifying is conducted using a method selected from the group consisting of Polymerase Chain Reaction, Ligase Chain Reaction, Self-Sustained Sequence Replication, Strand Displacement Amplification, Transcriptional Amplification System, Q-Beta Replicase, Nucleic Acid Sequence Based Amplification, Cleavage Fragment Length Polymorphism, Isothermal and Chimeric Primer-initiated Amplification of Nucleic Acid, and Ramification-extension Amplification Method.

52. The method according to claim 46, wherein the amplifying, the hybridizing and the contacting are simultaneously or sequentially carried out.

53. The method according to claim 46, further comprising cultivating the sample containing Listeria spp. in an enrichment medium before the amplifying, to enhance growth of the Listeria spp.

54. The method according to claim 53, wherein the enrichment medium contains, per 1 L of distilled water, about 10 to about 40 g of tryptic soy broth, about 1 to about 10 g of yeast extract, and about 1 to about 10 g of lithium chloride.

55. The method according to claim 54, wherein the enrichment medium further comprises at least one component selected from the group consisting of about 1 to about 10 g of beef extract, and/or a vitamin mix containing about 0.01 to about 0.5 mg of riboflavin, about 0.5 to about 1.5 mg of thiamine and about 0.01 to about 1.5 mg of biotin; about 1 to about 5 g of pyruvate or a salt thereof; and about 0.01 to about 1 g of ferric ammonium citrate.

56. The method according to claim 55, wherein the enrichment medium further comprises a buffer compound.

57. The method according to claim 56, wherein the buffer compound comprises 3-(N-morpholino)propanesulfonic acid (MOPS) and a sodium salt thereof.

58. The method according to claim 57, wherein the buffer compound comprises about 4 g of MOPS and about 7.1 g of sodium MOPS.

59. The method according to claim 53, wherein the enrichment medium comprises about 1 to about 10 mg of acriflavine, about 5 to about 15 mg of polymyxin B, and about 10 to about 30 mg of ceftazidime.

60. The method according to claim 53, wherein the enriched medium comprises, per 1 L of distilled water, about 10 to about 40 g of tryptic soy broth, about 1 to about 10 g of yeast extract, about 1 to about 10 g of lithium chloride; about 1 to about 10 g of beef extract and/or a vitamin mix containing about 0.01 to about 0.5 mg of riboflavin, about 0.5 to about 1.5 mg of thiamine, and about 0.01 to about 1.5 mg of biotin; about 1 to about 5 g of pyruvate or a salt thereof; about 0.1 to about 1 g of ferric ammonium citrate; about 4 g of 3-(N-morpholino)propanesulfonic acid (MOPS) and about 7.1 g of sodium MOPS; and about 1 to about 10 mg of acriflavine, about 5 to about 15 mg of polymyxin B, and about 10 to about 30 mg of ceftazidime.

61. The method according to claim 53, wherein the enrichment medium does not comprise one of esculin or peptone, or both.

62. The method according to claim 53, wherein the enriched medium comprises, per 1 L of distilled water, about 30 g of tryptic soy broth, about 6 g of yeast extract, about 1 to about 10 g of lithium chloride; about 5 g of beef extract and/or a vitamin mix containing about 0.1 mg of riboflavin, about 1.0 mg of thiamine, and about 1.0 mg of biotin; about 2 g of sodium pyruvate; about 0.2 g of ferric ammonium citrate; about 4 g of 3-(N-morpholino)propanesulfonic acid (MOPS) and about 7.1 g of sodium MOPS; and about 5 mg of acriflavine, about 10 mg of polymyxin B, and about 20 mg of ceftazidime.

63. The method according to claim 53, wherein the enrichment medium is a tryptic soy broth supplemented with a yeast extract, or a brain-heart infusion broth.

64. The method according to claim 46, wherein the sample is food or a surface wipe.

65. The kit according to claim 11, further comprising
(d) a reverse transcriptase activity for reverse transcription of the target Listeria spp.
(e) an amplifying activity for a PCR amplification of the target DNA sequence to produce a Listeria spp. PCR fragment; and
(f) an RNase H activity.

66. The kit according to claim 65, further comprising positive, internal, and negative controls.

67. The kit according to claim 66, further comprising uracil-N-glycosylase.

68. The kit according to claim 65, wherein the probe is coupled to a detectable label at both of its 3′- end and 5′-end.

69. The kit according to claim 68, wherein the detectable label is a fluorescent label.

70. The kit according to claim 69, wherein the probe is labeled with a FRET pair.

71. The kit according to claim 65, wherein the probe is linked to a solid support.

72. The kit according to claim 65, which further comprises an amplification buffer.

73. The kit according to claim 65, which further comprises an amplifying polymerase activity.

74. The kit according to claim 65, wherein the RNase H activity is the activity of a thermostable RNase H.

75. The kit according to claim 65, wherein the RNase H activity is a hot start RNase H activity.

76. The kit according to claim 65, wherein the first primer is one or more selected from the group of oligonucleotides of SEQ ID NOs: 1-3:

| | |
|---|---|
| CCAAGCAGTGAGTGTGAGAAG, | (SEQ ID NO: 1) |
| CCAAGCAGTGAGTGTGAGAA, | (SEQ ID NO: 2) |
| and | |
| TCCAAGCAGTGAGTGTGAGAA. | (SEQ ID NO: 3) |

77. The kit according to claim 65, wherein the second primer is one or more selected from the group of oligonucleotides of SEQ ID NOs: 5-9:

| | |
|---|---|
| TGACAGCGTGAAATCAGGAAC, | (SEQ ID NO: 5) |
| TTGACAGCGTGAAATCAGG, | (SEQ ID NO: 6) |
| TGACAGCGTGAAATCAGGA, | (SEQ ID NO: 7) |
| TGACAGCGTGAAATCAGGA, | (SEQ ID NO: 8) |
| and | |
| GACAGCGTGAAATCAGGA. | (SEQ ID NO: 9) |

78. The kit according to claim 65, wherein the probe comprises the sequence of SEQ ID NO: 21 or SEQ ID NO: 22:
TGCGAAGACTGAGCTGTGATGG (SEQ ID NO: 21), wherein at least one of nucleotides at positions 8 and 9 are a ribonucleotide, and
CCATCACAGCrUrCrArUGCTTCGC (SEQ ID NO. 22), wherein at least one of “rU,” “rC,” “rA,” and “rU” at positions 11, 12, 13, and 14, respectively, is a ribonucleotide.

79. The kit according to claim 65, wherein the probe is one or more selected from the group consisting of oligonucleotides of SEQ ID NOs: 10-14:
TGCGAAGCrATGAGCTGTGATGG (SEQ ID NO: 10), wherein “rA” at position 9 is a ribonucleotide,
TGCGAAGrCATGAGCTGTGATGG (SEQ ID NO: 11), wherein “rC” at position 8 is a ribonucleotide,
CCATCACAGCTCArUGCTTCGC (SEQ ID NO: 12), wherein “rU” at position 14 is a ribonucleotide,
CCATCACAGCTrCrArUGCTTCGC (SEQ ID NO: 13), wherein “rC,” “rA,” and “rU” at positions 12, 13, and 14, respectively, are a ribonucleotide; and
CCATCACAGCrUrCrArUGCTTCGC (SEQ ID NO: 14), wherein “rU,” “rC,” “rA,” and “rU” at positions 11, 12, 13, and 14, respectively, are a ribonucleotide.

80. The kit according to claim 12, wherein the probe is in free form.

81. The kit according to claim 65, wherein the probe is in free form.
